(19) United States
(12) Patent Application Publication
(10) Pub. No.: US 2006/0193860 A1
(43) Pub. Date: Aug. 31, 2006

(54) Treatment of schizophrenia

(76) Inventors: Boris Skurkovich, Pawtucket, RI (US); Simon Skurkovich, Rockville, MD (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 11/415,719

(22) Filed: May 2, 2006

Related U.S. Application Data
(63) Continuation of application No. 10/603,507, filed on Jun. 25, 2003.
Application No. 10/603,507 is a continuation of application No. 10/422,119, filed on Apr. 24, 2003, now abandoned.
(60) Provisional application No. 60/411,925, filed on Sep. 18, 2002.

(51) Int. Cl. A61K 39/395

(52) U.S. Cl. 424/145.1

(57) ABSTRACT

The invention includes methods of treating schizophrenia in a patient where the method includes administration of antibodies to TNF-alpha and interferon-gamma to the patient. The invention further includes methods of treating schizophrenia in a patient where the method comprises administration of an antibody to TNF-alpha.

### BACKGROUND OF THE INVENTION

The ability of the immune system to discriminate between “self” and “non-self” antigens is vital to the functioning of the immune system as a specific defense against invading microorganisms. “Non-self” antigens are those antigens on substances entering or present in the body which are detectably different or foreign from the animal's own constituents, whereas “self” antigens are those which, in the healthy animal, are not detectably different or foreign from its own constituents. However, under certain conditions, including in certain disease states, an individual's immune system will identify its own constituents as “non-self,” and initiate an immune response against “self” material, at times causing more damage or discomfort as from an invading microbe or foreign material, and often producing serious illness in an individual. Autoimmune disease results when an individual's immune system attacks his own organs or tissues, producing a clinical condition associated with the destruction of that organ or tissue, as exemplified by diseases such as rheumatoid arthritis, insulin-dependent diabetes mellitus, acquired immunodeficiency syndrome (“AIDS”), hemolytic anemias, rheumatic fever, Crohn's disease, Guillain-Barré syndrome, psoriasis, thyroiditis, Graves' disease, myasthenia gravis, glomerulonephritis, autoimmune hepatitis, multiple sclerosis, systemic lupus erythematosus, dystrophic epidermolysis bullosa, and the like. Blocking, neutralizing or inhibiting the immune response or removing its cause in these cases is, therefore, desirable.

Autoimmune disease may be the result of a genetic predisposition alone or as the result of the influence of certain exogenous agents such as, viruses, bacteria, or chemical agents, or as the result of the action of both. Some forms of autoimmunity arise as the result of trauma to an area usually not exposed to lymphocytes, such as neural tissue or the lens of the eye. When the tissues in these areas become exposed to lymphocytes, their surface proteins can act as antigens and trigger the production of antibodies and cellular immune responses which then begin to destroy those tissues. Other autoimmune diseases develop after exposure of the individual to antigens which are antigenically similar to, that is cross-reactive with, the individual's own tissue. For example, in rheumatic fever an antigen of the streptococcal bacterium, which causes rheumatic fever, is cross-reactive with parts of the human heart. The antibodies cannot differentiate between the bacterial antigens and the heart muscle antigens, consequently cells with either of those antigens can be destroyed.

Other autoimmune diseases, for example, insulin-dependent diabetes mellitus (involving the destruction of the insulin producing beta-cells of the islets of Langerhans), multiple sclerosis (involving the destruction of the conducting fibers of the nervous system), and rheumatoid arthritis (involving the destruction of the joint lining tissue), are characterized as being the result of a mostly cell-mediated autoimmune response and appear to be due primarily to the action of T-cells (See, Sinha et al., Science 248:1380 (1990)). Yet others, such as myesthenia gravis and systemic lupus erythematosus, are characterized as being the result of primarily a humoral autoimmune response (Sinha et al., Science 248:1380 (1990)). As an example, dystrophic epidermolysis bullosa has been attributed to mutations in the non-collagenous domains of collagen type VII. These mutations result in the lack of formation of the normal anti-parallel collagen type VII dimers. The mutated collagen forms epitopes recognized as “non-self” by the immune system, and therefore autoantibodies are generated, resulting in the rapid degeneration of the basement membrane of the skin (Chen, et al., J. Biol. Chem. 276: 21649 (2001)). Similarly, pemphigus vulgaris is attributed to the presence of auto-antibodies to desmosomes, specifically the desmoglein 3 protein, which are the points of intracellular contact between epithelial cells. The auto-antibodies destroy the adhesion between cells, resulting in a loss of epithelial integrity and elasticity. Nevertheless, the autoimmune diseases share a common underlying pathogenesis, resulting in the need for safe and effective therapy. Yet none of the presently available drugs are completely effective for the treatment of autoimmune disease, and most are limited by severe toxicity.

In recent years, a new point of view on the pathogenesis of autoimmune diseases, including AIDS, has developed, in which it has been suggested that autoimmune disease is connected with a disturbance in the synthesis of interferons (IFNs) and other cytokines induced by interferons (Skurkovich et al., Nature 217:551-2 (1974); Skurkovich et al., Annals of Allergy 35:356 (1975); Skurkovich et al., J. IFN Res. 12, Suppl. 1: S110 (1992); Skurkovich et al., Med. Hypoth. 41:177-185 (1993); Skurkovich et al., Med. Hypoth. 42:27-35 (1994); Gringeri et al., Cell. Mol. Biol. 41(3):381-387 (1995); Gringeri et al., J Acquir. Immun. Defic. Syndr. 13:55-67 (1996)). IFN has been found in the circulation of patients with autoimmune diseases, and it has been neutralized in vivo with antibody to leukocyte (alpha) IFN (“IFNα”). Healthy people do not have interferon in their blood (Skurkovich et al., 1975). In addition, it has been shown that hyperproduced alpha IFN is found not only in the circulation of patients with classic autoimmune diseases, but also in patients with HIV infection (DeStefano et al., J. Infec. Disease 146:451 (1982)), where its presence is a predictive marker of AIDS progression (Vadhan-Raj et al., Cancer Res. 46:417 (1986)). The IFN induced by HIV has low anti-(HIV) viral activity (Gendelman et al., J. Immunol. 148:422 (1992)). It was shown that the circulating alpha IFN possesses antigenic specificity like natural alpha IFN, which is pH stable, but this interferon is pH labile like gamma IFN (Preble et al., Science 216:429 (1982)); thus, it is known as aberrant alpha IFN.

Investigators have also shown that tumor necrosis factors (TNF alpha and TNF beta) also play a significant role in the pathology of autoimmune diseases. For example, the presence of TNF alpha has been correlated with rheumatoid arthritis (RA) (Brennan et al., Brit. J. Rheum. 31(5):293-8 (1992)), and TNF alpha has been found to be related to an increase in the severity of collagen induced arthritis in animal models (Brahn et al., Lymphokine and Cytokine Res. 11(5):253 (1992)), while it has also been shown that anti-TNF alpha antibody administration ameliorates collagen induced arthritis (Williams et al., Clin. & Exp. Immunol. 87(2):183 (1992)). TNF alpha is increased in the serum of RA patients (Holt et al., Brit. J. Rheum. 21(11):725 (1992); Altomonte et al., Clin. Rheum. 11(2):202 (1992), and both the cytokine (Chu et. al., Brit. J. Rheum. 31(10):653-661 (1992)) and its receptors have been identified in rheumatoid synovium, as well as at the cartilage-pannus junction (Deleuran et al., Arthritis Rheum. 35(10):1180 (1992)).

In addition, increased circulating levels of TNF alpha have been found to be associated with disease progression in patients with multiple sclerosis (Shariff et al., N. Engl. J. Med. 325(7):467-472 (1992)); while increased serum levels of soluble TNF receptor and gamma interferon (“gamma-IFN”) have been independently correlated with disease activity in individuals, e.g., those with systemic lupus erythematosus (Aderka et. al., Arthritis Rheum. 36(8):1111-1120 (1993); Machold et al., J. Rheumat. 17(6):83 1-832 (1990)). The spontaneous release of interferon and TNF in HIV-positive subjects (Vilcek et al., In AIDS: The Epidemic of Karposi's Syndrome and Opportunistic Infections, A. E. Friedman-Kien & L. J. Laubenstein, eds. Masson Publishing, New York, N.Y., 1986; Hess et al., Infection 19, Suppl 2:S93-97 (1991); Biglino et al., Infection 19(1):11/7-11/17 (1991)), and the decline seen in the serum levels of TNF alpha in RA patients following long term administration of the disease modifying drug sulfasalazine (Danis et al., Ann. Rheum. Disease 51(8):946 (1992)), further suggest that the concentrations of cytokines and/or their receptors is reflected in the clinical course of autoimmune disease.

IFN is known to induce tumor necrosis factor (TNF) and its receptors (Lau et al., AIDS Research and Human Retroviruses 7:545 (1991)), which enhances virus replication (Matsuyama et al., Proc. Natl. Acad. Sci. USA 86:2365 (1989)). In addition to its presence in the circulation, IFNs have also been found in the cerebrospinal fluid in some patients with psychiatric mid neurologic diseases (Lebikova et al., Acta Biol. Med. Germ. 38:879 (1979); Preble et al., Am. J. Psychiatry 142:10 (1985)), as well as in patients with rheumatoid arthritis. Therefore, since healthy people do not have interferons in their spinal or synovial fluids, the inventors have suggested that one or more alpha IFNs may be involved in the development of the initial autoimmune disease response. Consequently, the removal and/or neutralization of alpha IFN has been proposed as a method of treatment of patients with auto immune disease, including AIDS. The appearance of cytokines and autoimmunogens induced by alpha IFN and their prolonged circulation in the body is an inseparable part of the development of autoimmune disease, triggering immune dysregulation in autoimmune disease, including AIDS. See, U.S. Pat. Nos. 4,824,432; 4,605,394; and 4,362,155, herein incorporated by reference. However, it now appears that gamma IFN also plays a pathogenetic role since each participates in immune regulation.

In addition to classic autoimmune disease and AIDS, autoantibodies play a pathogenic role in many other pathological conditions. For example, after cell (or organ) transplantation or after heart attack or stroke, certain antigens from the transplanted cells (organs) or necrotic cells from the heart or the brain can stimulate the production of autoantibodies or immune lymphocytes (Johnson et al., Sem. Nuc. Med. 19:238 (1989); Leinonen et al., Microbiol. Path. 9:67 (1990); Montalban et al., Stroke 22:750 (1991)), which later participate in rejection (in the case of a transplant) or attack cardiac or brain target cells, aggravating the condition. Moreover, in human autoimmune disease certain cells express abnormally elevated levels of HLA class II antigens, which is stimulated by the disturbed production of cytokines, e.g., gamma IFN alone, or gamma IFN in combination with TNF (Feldman et al., “Interferons and Autoimmunity,” In IFN 9, Academic Press, p. 75 (1987).

Recognition of the important role of cytokines in autoimmune disease has fostered the development of a new generation of therapeutic agents to modulate cytokine activity. Preliminary results of trials in which anti-interferon polyclonal antibodies were administered to a small group of rheumatoid patients suggest improvement in both the clinical and the laboratory manifestations of the disease (Skurkovich et al., Annals of Allergy 39:344-350 (1977)). Moreover, proteins, such as polyclonal antibodies and soluble receptors targeted against interferons and TNF-α are currently being evaluated in clinical trials for the treatment of RA and other autoimmune diseases. The administration of monoclonal antibodies to TNF-α has provided encouraging early results in the treatment of patients with severe RA (Elliott et. al., J. Cell. Biochem., Suppl 17B: 145 (1993); Elliott et al., Lancet 344:1105-1110 (1994)). Also positive preliminary results were achieved in AIDS patients given antibodies or other agents to reduce the level of circulating alpha IFN in the body (Skurkovich et al., 1994; Gringeri et al., 1996). However, because autoimmune diseases are complex, often characterized by multiple cytokine abnormalities, effective treatment appears to require the simultaneous administration or utilization of several agents, each targeting a specific cytokine pathway or its by-product. To meet this need, the methods of treatment of the present invention include not only the use of specific antibodies, but also provide pleiotrophic autoimmune inhibitors, including antibodies to cytokines and HLA class II antigens, and antigens for the removal of autoantibodies to target cells or DNA. The use of these antibodies and antigens as disclosed in the present invention results in the removal, neutralization or inhibition of the pathogenic cytokine(s), HLA class II antigens, and/or autoantibody(ies) to target cells or DNA from the autoimmune patient, thereby significantly improving the quality of life of the individual.

### SUMMARY OF THE INVENTION

The present invention includes a method of treating schizophrenia in a patient. The method comprises administering to the patient an effective amount of an antibody to tumor necrosis factor-alpha and an effective amount of an antibody to interferon-gamma.

In one aspect, the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

In another aspect, the antibody is administered by the route selected from the group consisting of intramuscularly, intravenously, intradermally, cutaneously, ionophoretically, topically, locally, and inhalation.

In yet another aspect, the antibody is selected from the group consisting of a polyclonal antibody, a monoclonal antibody, a synthetic antibody, a heavy chain antibody and a humanized antibody.

In still another aspect, the heavy chain antibody is selected from the group consisting of a camelid antibody, a heavy chain disease antibody, and a variable heavy chain immunoglobulin.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a diagram depicting TNF-alpha levels and PANSS (Positive and Negative Syndrome Scale) scores in a patient with schizophrenia before and after treatment with anti-cytokine therapy. The diagram depicts TNF-alpha levels measured in the blood of the patient on days 1-34 and selected PANSS scores on days 0, 12, and 34. Anti-cytokine therapy was administered on days 1-5.

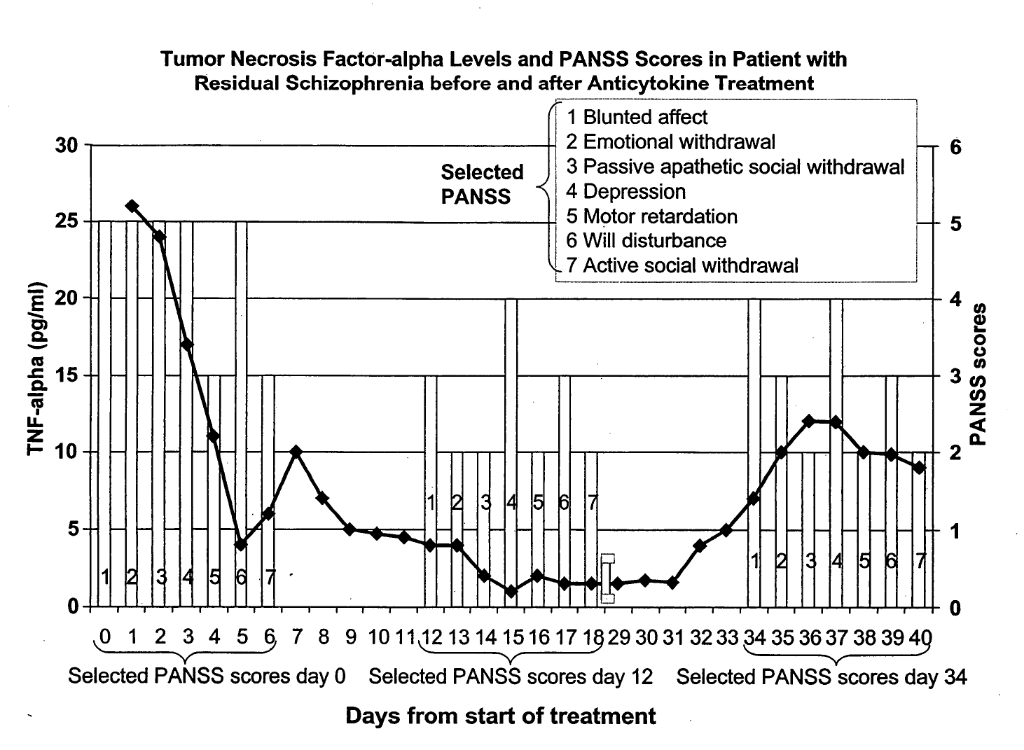

### DETAILED DESCRIPTION OF THE INVENTION

The present invention provides a method of treating autoimmune-based psychological diseases including, but not limited to, schizophrenia, by blocking, neutralizing, or inhibiting tumor necrosis factor-alpha (TNF-alpha) in a patient having the disease. The invention also provides a method of treating autoimmune-based psychological diseases, including, but not limited to, schizophrenia, by blocking, neutralizing or inhibiting TNF-alpha in combination with blocking, neutralizing or otherwise inhibiting IFN-gamma.

TNF-alpha is blocked, neutralized or inhibited by administering to a patient in need an effective amount of antibody to TNF-alpha. The antibody to TNF-alpha is a monoclonal antibody, a polyclonal antibody, or a combination of both. Alternatively, TNF-alpha is blocked, neutralized, or inhibited by administering to a patient in need an effective amount of a biologically active fragment of antibody to TNF-alpha, a functional equivalent of antibody to TNF-alpha, a derivative of an antibody to TNF-alpha, or an allelic or species variant of antibody to TNF-alpha. Humanized antibodies to TNF-alpha are also included in the present invention, including those described in U.S. Pat. No. 6,329,511 to Vasquez, et al. (assigned to Protein Design Labs, Inc. (Fremont, Calif.)), which is incorporated herein by reference. The present invention further contemplates the use of heavy chain antibodies, including, but not limited to antibodies derived from camelid species, and other heavy chain antibodies as detailed extensively elsewhere herein. Preparation of antibodies which are useful in the present invention is more fully discussed below.

IFN-gamma is blocked, neutralized or inhibited by administering to a patient in need an effective amount of antibody to IFN-gamma. The antibody to IFN-gamma is a monoclonal antibody, a polyclonal antibody, or a combination of both. Alternatively, IFN-gamma is blocked, neutralized, or inhibited by administering to a patient in need an effective amount of a biologically active fragment of antibody to IFN-gamma, a functional equivalent of antibody to IFN-gamma, a derivative of an antibody to IFN-gamma, or an allelic or species variant of antibody to IFN-gamma. Humanized antibodies to IFN-gamma are also included in the present invention, including those described in U.S. Pat. No. 6,329,511 to Vasquez, et al. (assigned to Protein Design Labs, Inc. (Fremont, Calif.)), which is incorporated herein by reference. The present invention further contemplates the use of heavy chain antibodies, including, but not limited to antibodies derived from camelid species, and other heavy chain antibodies as detailed extensively elsewhere herein. Preparation of antibodies which are useful in the present invention is more fully discussed below.

Schizophrenia is one of the most debilitating and emotionally devastating diseases known to humans. Schizophrenia has known biological basis, and is not the result of traumatic psychological or emotional disturbances. Recent evidence further supports that autoimmune dysfunction plays a role in the etiology of schizophrenia (Gaughran, 2002, Int. Rev. Neurobiol. 52:275-302). In addition, IFN and TNF-alpha level abnormalities have been reported in schizophrenic patients (Libikova et al., 1979, Acta Biologica et Medica Germanica 38: 879-893; Naudin et al., 1997, Schizophrenia Research 26: 227-233; Arolt et al., 2000, Molecular Psychiatry 5: 1508). Approximately 1 to 1.5% of all Americans will be afflicted with schizophrenia at some point in their life.

Schizophrenia is characterized by a number of distinctive and predictable symptoms. Positive symptoms are most commonly associated with the disease, and are characterized by grossly abnormal behavior such as thought disorder, delusions, and hallucinations. Thought disorder is the diminished ability to think clearly and logically, and often manifests as disconnected and nonsensical language that renders the person with schizophrenia incapable of participating in conversation, contributing to his alienation from his family, friends, and society. Delusions are common among individuals with schizophrenia. An affected person may believe that he is being conspired against (paranoid delusion). Broadcasting delusions describe a type of delusion in which the individual believes that his or her thoughts can be heard by others. Hallucinations can be heard, seen, or even felt; most often they take the form of voices heard only by the afflicted person. Less obvious than the “positive symptoms” but equally serious are the deficit or negative symptoms that represent the absence of normal behavior. These include flat or blunted affect (i.e. lack of emotional expression, apathy, and social withdrawal).

Schizophrenia is somewhat more common in those persons who are genetically predisposed to the disease. The first psychotic episode generally occurs in late adolescence or early adulthood. The probability of developing schizophrenia as the offspring of two parents, neither of whom has the disease, is 1 percent. The probability of developing schizophrenia as the offspring of one parent with the disease is approximately 13 percent, and the probability of developing schizophrenia as the offspring of both parents with the disease is approximately 35 percent. Approximately 75% of persons with schizophrenia develop the disease between 16 and 25 years of age. Onset is uncommon after age 30, and rare after age 40.

To ensure the safety of the affected person and to allow monitoring and evaluation of drug treatments, hospitalization is often necessary in cases of acute schizophrenia. Antipsychotic drugs (also called neuroleptics) are currently the most common treatment for people with schizophrenia. Aside from the importance of treating the autoimmune nature of the aforementioned disease, treatment of these diseases is important to improving social interactions and emotional well-being of patients afflicted with this disease.

Interferons are now known to be not only an antiviral and anti-proliferative cytokine, but it is also a factor which plays an important role in normal and pathological immunity. For the normal functioning of the immune system, it is necessary for an individual to have a normally functioning cytokine system. The interferon system in humans is a very stable system. Since healthy people do not have interferon in their blood, prolonged hyperproduction of interferon—alpha and/or gamma interferons—typically indicate the presence of immune disease.

Upon observation of the diverse clinical pictures manifested in patients with various autoimmune disease, which includes hypersensitivity of the immediate type (e.g., bronchial asthma, which is also an autoimmune condition), and AIDS (a viral disease with autoimmune components), it becomes apparent that these diseases have in common a large number of similar laboratory characteristics. This suggests that a similar disease mechanism is occurring in each autoimmune disease, but in different target cells. Thus, it is the unique target (e.g., skin, joints, liver, central nervous system (CNS), and the like) of each autoimmune disease that leads to its characterization in terms of clinical manifestations. For example, an autoimmune attack destroying the insulin producing beta-cells of the islets of Langerhans of an individual would be diagnosed as diabetes (Type I), whereas autoimmune destruction of the conducting fibers of the nervous system is characteristic of multiple sclerosis, or autoimmune destruction of the joint lining tissue is characteristic of rheumatoid arthritis. Likewise in the case of skin transplantation, the skin area can be damaged. Yet in each case, the mechanism underlying the autoimmune response is similar; a high level of IFNs, a detectable level of TNF, an elevated level of HLA class II antigens in the blood or on the surface of the cells, and antibodies to target cells. In addition, cells taken from autoimmune patients show a decreased production of IFNs in vitro, even after stimulation with an interferonogen. Consequently, the method of treatment of the various autoimmune diseases is similar in principle, despite the apparent clinical differences among the diseases.

The present invention is based upon the findings that the optimal treatment of each different autoimmune disease or autoimmune condition involves the removal, neutralization or inhibition of complex pathological agents (including hyperproduced cytokines) from the patient, and/or the administration to the patient of an effective amount of selected molecules or antibodies, or their receptors, to bind to, neutralize or inhibit the circulating pathological agents and/or those on the surface of the cells targeted in the specific autoimmune response (“target cells”). One indicator of an autoimmune disease is the hyperproduction of IFN-alpha or, to be more exact, the disturbance of the synthesis of one or more alpha IFNs (alpha IFN comprises at least 15 distinct subtypes). In most patients with autoimmune disease, some level of gamma IFN is also found. Patients with systemic lupus erythematosus (“SLE”) and AIDS appear to have the highest levels of alpha IFN, as compared with patients with other autoimmune diseases (See, Skurkovich et al., Annals of Allergy 35:356 (1975); DeStefano et al., 1982).

Alpha IFN is secreted by somatic cell and leukocytes, accumulating on the membranes of cells and entering the bloodstream. In autopsies, alpha IFN has been found, for example, on the surface of cells in the pancreas of patients with insulin dependent diabetes (Foulis et. al, Lancet 2:1423 (1987)), in skin lesions of patients with psoriasis (Livden et. al., Arch Dermalot Res. 281:392 (1989)), on the surface of brain cells of patients with the psychiatric complications of systemic lupus erythematosus (“SLE”) ((Shiozawa et al., Arthr. Rheum. 35:417 (1992)), and in the circulating body fluids of animal and human patients with autoimmune disease ((Skurkovich et al., 1975; DeStefano et al., 1982). For instance, alpha IFN has been found circulating in the blood of autoimmune NZB/W and mrl/lpr mice (Skurkovich et al., Ann. Internat'l Congress for Interferon Research (1981), and in the circulation of patients with RA, SLE, Sjogren's syndrome, scleroderma, insulin-dependent diabetes, bronchial asthma, AIDS, and other autoimmune diseases (Skurkovich et. al., 1975; Hooks et al., N Engl. J. Med 301:5 (1979); DeStefano et al., 1982). Of particular interest is a recent discovery that interferon is also found in the blood and spinal fluid of patients with neurological diseases, including, e.g., schizophrenia (Lebikova et al., Med Microbiot Immun. 166:355 (1978); Preble et al., 1985), depression, and multiple sclerosis (Link et al., Ann. Neurol 36:379 (1994)).

The uninterrupted production of alpha IFN is apparently connected with the weakening or absence of the alpha IFN repressor. In general, hyperproduction of alpha IFN is an indicator of immunological disintegration, and many scientists consider alpha IFN to be a recognized marker of the presence of an autoimmune condition ((Skurkovich et al., 1975; Hooks et al., 1979). Moreover, the disturbance of alpha IFN production in an individual changes the biological activity of the cells, bringing about the production of autoantigens (Skurkovich et al., 1994; Shattner et al., Am. J. Med Sci. 295:532 (1988)). The hyperproduction of alpha IFN also stimulates the production of tumor necrosis factor and its receptors, particularly TNF-alpha (Lau et al., 1991). Increased production of autoantigens leads to the activation of the T-cells, and to the production of gamma IFN. It is possible every autoantigen stimulates the induction of a unique, specific gamma IFN.

In addition, in human autoimmune disease some cells express abnormally elevated levels of HLA class II antigens, or in some cases HLA class I or III antigens, which is stimulated by the disturbed production of gamma IFN, alone or in combination with TNF (Feldman et. al., 1987). This synthesis of HLA class II antigens (or HLA class I or III antigens) plays an important role in the pathogenesis of autoimmune disease and AIDS. The disturbance of the production of HLA class II antigen in an individual leads to a pathological disturbance of the presentation of antigens to the T-cells, to disrupted T/B cooperation, and to the dysregulation of the interactions among T-cells.

Every antigen is an interferonogen; “self” cannot induce IFN. Thus, the production of IFN signals the invasion by a foreign antigen, or in this case the presence of an autoantigen. The production of IFN and its prolonged circulation in the body is an inseparable part of the development of autoimmune disease, and triggers immunological chaos. For example, antibodies to CD4 in patients with HIV infection (Dorsett et al., Am. J. Med 78:62 1 (1985)) can cross-react with HLA class II antigen, which in turn are induced by gamma IFN, or by gamma IFN in combination with TNF, and possibly by alpha IFN, which induces TNF.

Alpha IFN and gamma IFN are biologically dangerous elements in certain people. If injected into a human or animal having a genetic predisposition to develop an autoimmune disease, the interferons can trigger or exacerbate the autoimmune disease in the recipient. For example, administration of alpha IFN, gamma IFN, or an inducer of alpha IFN to autoimmune NZB/W and MRL/lpr/lpr mice have resulted in an aggravation of the autoimmune response in the animal, augmented morbidity, and increased mortality (Carpenter et al., Lab Invest. 23:628 (1970); Engleman et al., Arthr. Rheum. 24:1396 (1981); Heremans et al., Infect Immun. 21:925(1978)). Injection of one unit of recombinant gamma-IFN into the thyroid gland of CBA mice caused autoimmune thyroiditis (Remy et al., Immunol. Today. 8:73 (1987)). Administration of alpha IFN to human patients with psoriasis (a disease with an autoimmune component) was found to exacerbate, rather than alleviate the clinical manifestations of the disease (Quesada et al., Lancet 2:1466 (1986)). Injection of natural or recombinant alpha IFN, and sometimes gamma IFN, to cancer patients has reportedly triggered or exacerbated autoimmune parotitis, epididymitis, and thyroiditis, SLE, RA, Graves' disease, and other autoimmune conditions (See, e.g., Quesada et al., Clin. Oncol. 2:4234 (1986); Bevan et al., Lancet 2:561 (1985); Ronnblom, et al. J Intern. Med 227:207 (1990); Conlon et al., (Cancer 65:2237 (1990); Machold et al., J. Rheum. 17:831 (1990); Schilling et al., Cancer 68:1536 (1991); Ronnblom et al., Ann. Intern. Med 115:178 (1991)). Alpha IFN injections in patients with different types of viral hepatitis have induced autoimmune hepatitis (See, e.g., Ohta et al., J Gastroenterol. 88:209 (1991); Fattovich et al., Brit. J. Med. Virol. 34: 132 (1991)). In addition, it has been reported that a patient with multiple sclerosis (“MS”) given recombinant alpha IFN subcutaneously (Larrey et al., JAMA 261:2065 (1989)), and another given recombinant gamma IFN (Paniteh et al., Lancet 1:893 (1987)) intrathecally, manifested clinical relapses at rates significantly higher than expected.

On the other hand, the neutralization of individual cytokines, such as alpha IFN or TNF-alpha, from the blood has been associated with a significant therapeutic effect, in patients with RA and in patients with AIDS (Skurkovich et al., 1975; Gringeri et al., 1996). It is a purpose of the present invention to provide methods of treating autoimmune disease by the use of pleiotrophic autoimmune inhibitors, acting on each of the known aberrant cytokine pathways in the patient and/or removing pathogenic cytokines, HLA antigens, or autoantibodies from the autoimmune patient.

The terms “patient” and “individual” are interchangeably used to mean a warm-blooded animal, such as a mammal, suffering from a disease, such as an autoimmune disease or “graft versus host” disease, or is in danger of rejection of a transplanted allogeneic tissue or organ. It is understood that humans and animals are included within the scope of the term “patient” or “individual.”

The articles “a” and “an” are used herein to refer to one or to more than one (i.e. to at least one) of the grammatical object of the article. By way of example, “an element” means one element or more than one element.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies, heavy chain antibodies, camelid antibodies, fragments thereof, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

“Cytokines” are intercellular mediators secreted by the lymphocytes and/or macrophages. For example, cytokines play a role in the generation of an immune response, such as in an immune response to an infection or infectious organism. Cytokines including, for example, interferons (alpha IFN and gamma IFN) and TNFs induce other cytokines which participate in the development of different autoimmune conditions and diseases. In the development of anti-cytokine therapy in accordance with the present invention, considerable emphasis has been placed on these three cytokines, because it appears that by neutralizing these key cytokines (alpha IFN, gamma IFN and TNF), it is possible to decrease, halt or prevent the synthesis of the cytokines induced by them. However, is certain autoimmune conditions or diseases, including IDDM and SLE, the induction of another cytokine (interleukins, specifically IL-6) is so great and exerts such a strong pathological influence, that it is desirable to remove IL-6 together with the other cytokines.

IL-6 is made by several cells, including T-cells, B-cells, and others (Hirano et al., Clin. Immunol 62:560 (1992)), and induces insulinitis in IDDM. In response to gamma IFN and TNF, B-cells of the pancreas produce large quantities of IL-6. It is also an important pathological factor in the pathogenesis of SLE, where is has been found to be present at a high level. IL-6 stimulates differentiation in B-cells and hyperactivity of T-cells (Snick et al., Ann. Rev. Immunol. 8:253 (1990)). The increase in IL-6 parallels the increase of TNF-alpha (Majer et. al., Lupus 2:359-365 (1993)).

The term “autoimmune inhibitor” is used to refer to a “compound” or “compounds,” including one or more molecules, antigens, and/or antibodies (alone or in combination), which when administered in an effective amount to a patient, binds to, neutralizes or inhibits circulating pathological agents and/or those on the surface of target cells, and which when placed in extracorporeal contact with the patient's body fluids effects the removal, neutralization or inhibition of complex pathological agents (including hyperproduced cytokines and autoantibodies). The autoimmune inhibitor may also comprise antibodies to a receptor of the autoantigen. A “receptor” is a protein found on the surface of a target cell or in its cytoplasm, that has a binding site with high affinity to a particular signaling substance (e.g., a cytokine, hormone, neurotransmitter, etc.). By competitively inhibiting the availability of the receptor with an analog or antibody to the receptor, the immune response to the autoimmunogen is modified or neutralized.

In accordance with the present invention, treatments involving administration of an autoimmune inhibitor to a patient, and treatments involving the extracorporeal exposure of the patient's fluid to an autoimmune inhibitor, may be performed alone or in combination.

Administered autoimmune inhibitor of the invention binds to, neutralizes and/or inhibits the molecule(s) associated with or causing the autoimmune response in the patient. More specifically, administration of the autoimmune inhibitor to a patient results in suppression of pathological humoral and adaptive immunity in the patient. In other words, in accordance with the method of the present invention, treatment of a patient with the autoimmune inhibitor causes the humoral and adaptive immune response of the patient to be inhibited or neutralized over that which was, or would have been, present in the absence of treatment.

A patient is in need of treatment with an autoimmune inhibitor, when the patient is suffering from an autoimmune disease, or “graft-versus-host” disease, or when treatment is needed to prevent rejection of transplanted allogeneic tissues or organs, or when the patient has produced autoantibodies.

The term “autoimmune disease” refers to those disease states and conditions wherein the immune response of the patient is directed against the patient's own constituents, resulting in an undesirable and often terribly debilitating condition. As used herein, “autoimmune disease” is intended to further include autoimmune conditions, syndromes and the like. An “autoantigen” is a patient's self-produced constituent, which is perceived to be foreign or undesirable, thus triggering an autoimmune response in the patient, which may in turn lead to a chain of events, including the synthesis of other autoantigens or autoantibodies. An “autoantibody” is an antibody produced by an autoimmune patient to one or more of his own constituents which are perceived to be antigenic. For example, in AIDS disease the patient eventually produces autoantibodies to CD4 cells, in dystrophic epidermolysis bullosa, autoantibodies are produced to collagen, in pemphigus vulgaris, autoantibodies are produced to desmosomes and desmosome proteins desmoglein3 and desmoglein1, in SLE autoantibodies are produced to DNA, while in many other types of autoimmune disease autoantibodies are produced to target cells (see for example, Table I for examples of specific target cells of autoimmune disease).

Patients suffering from autoimmune diseases including, e.g., rheumatoid arthritis, insulin-dependent diabetes mellitus, hemolytic anemias, rheumatic fever, thyroiditis, Crohn's disease, myasthenia gravis, glomerulonephritis, autoimmune hepatitis, multiple sclerosis, dystrophic epidermolysis bullosa, systemic lupus erythematosus and others, are in need of treatment in accordance with the present invention. Treatment of patients suffering from these diseases by administration of autoimmune inhibitor and/or removal of compound(s) by extracorporeal immunosorption in accordance with the present invention will alleviate the clinical manifestations of the disease and/or minimize or prevent further deterioration or worsening of the patient's condition. Treatment of a patient at an early stage of an autoimmune disease including, e.g., rheumatoid arthritis, insulin-dependent diabetes mellitus, multiple sclerosis, myasthenia gravis, dystrophic epidermolysis bullosa, systemic lupus erythematosus, alopecia areata, vitiligo, psoriasis, or others, will minimize or eliminate deterioration of the disease state into a more serious condition.

For example, insulin-dependent diabetes mellitus (IDDM) is an autoimmune disease which is believed to result from the autoimmune response directed against the beta cells of the islets of Langerhans which secrete insulin. Treatment of a patient suffering from an early stage of IDDM prior to the complete destruction of the beta cells of the islets of Langerhans would be particularly useful in preventing further progression of the disease, since it would prevent or inhibit further destruction of the remaining insulin-secreting beta cells. It is understood that treatment of a patient suffering from an early stage of other autoimmune diseases will also be particularly useful to prevent or inhibit the natural progression of the disease state to more serious stages.

The method of the present invention is applicable to autoimmune diseases, such as those given in the following Table 1 (which is intended to be exemplary rather than inclusive).

TABLE 1
Autoimmune Diseases

| Disease | Tissue Affected |
|---|---|
| Addison's disease | adrenal |
| AIDS | immune system |
| Alopecia Areata | skin |
| Ankylosing Spondylitis | organs |
| Autoimmune diseases of the ear | ear |
| Autoimmune diseases of the eye | eye |
| Autoimmune hepatitis | liver |
| Autoimmune parotitis | parotid glands |
| Bone Marrow Transplant | Bone Marrow |
| Crohn's disease | intestine |
| Diabetes (Type I) | pancreas |
| Dystrohic epidermolysis bullosa | basement membranes of skin |
| Epididymitis | epididymis |
| Glomerulonephritis | kidneys |
| Graft/Transplant | throughout body |
| Graves' disease | thyroid |
| Guillain-Barré syndrome | nerve cells |
| Hashimoto's disease | thyroid |
| Hemolytic anemia | red blood cells |
| Juvenile rheumatoid arthritis | joints |
| Male infertility | sperm |
| Multiple sclerosis | nerve cells |
| Myasthenia Gravis | neuromuscular junction |
| Pemphigus | primarily skin |
| Psoriasis | skin |
| Psoriatic arthritis | joints |
| Rheumatic fever | heart and joints |
| Rheumatoid arthritis | joint lining |
| Sarcoidosis | multiple tissues and organs |
| Schizophrenia | CNS |
| Scleroderma | skin and connective tissues |
| Sjogren's syndrome | exocrine glands, and other tissues |
| Spondyloarthropathies | axial skeleton, and other tissues |
| Systemic lupus erythematosus | multiple tissues |
| Thyroiditis | thyroid |
| Uveitis | eyes |
| Vasculitis | blood vessels |
| Vitiligo | skin |

Autoimmune conditions for which the method of the present invention is applicable include, for example, AIDS, atopic allergy, bronchial asthma, dystrophic epidermolysis bullosa, eczema, Behcet's syndrome, leprosy, schizophrenia, inherited depression, transplantation of tissues and organs, chronic fatigue syndrome, Alzheimer's disease, Parkinson's disease, myocardial infarction, stroke, autism, epilepsy, Arthus's phenomenon, anaphylaxis, and alcohol and drug addiction. In the above-identified autoimmune conditions, the tissue affected is the primary target, in other cases it is the secondary target. These conditions are partly or mostly autoimmune syndromes. Therefore, in treating them, it is possible to use the same methods, or aspects of the same methods that are herein disclosed for treating autoimmune disease, sometimes in combination with other methods.

Preferred embodiments of the invention are directed toward the treatment of specific autoimmune disease or condition in a patient, including those identified herein, and particularly including schizophrenia, rheumatoid arthritis, dystrophic epidermolysis bullosa, systemic lupus erythematosus, multiple sclerosis, juvenile rheumatoid arthritis, and ankylosing spondylitis.

Patients who have received, or who are about to receive, an allogeneic tissue or organ transplant, such as an allogeneic kidney, liver, heart, skin, bone marrow, are also patients who are in need of prophylactic treatment with an autoimmune inhibitor and/or removal of compound(s) by extracorporeal immunosorption in accordance with the present invention. The autoimmune inhibitor of the present invention will minimize or prevent the adaptive and humoral immune response of the donor from rejecting the allogeneic tissue or organ of the donor. Likewise, for patients suffering from graft-versus-host disease treatment with an autoimmune inhibitor in accordance with the method of the present invention will minimize or prevent the adaptive and humoral immune response of the transplanted tissue or organ from rejecting the allogeneic tissue or organ of the donor.

Based on standard clinical and laboratory tests and procedures, an attending diagnostician, physician or other person skilled in the art, can readily identify those patients who are in need of treatment with an autoimmune inhibitor. Such an individual can also determine the compound or compounds to be included in the autoimmune inhibitor for treatment in accordance with the methods of the present invention, based upon the increased synthesis of cytokines typifying the general onset and progression of autoimmune disease, and on the clinical manifestations of the particular disease being treated.

The term “fluid” refers to blood, plasma, plasma containing leukocytes, serum, serum and leukocytes, peritoneal fluid, cerebrospinal fluid, synovial fluid, amniotic fluid, or the like, drawn from the patient in the practice of the present invention.

An effective amount of autoimmune inhibitor is that amount which is effective, upon single or multiple dose administration to a patient, to bind to, neutralize or inhibit the autoimmunogen(s) causing (directly or indirectly) or involved with the clinical manifestation(s) of the autoimmune disease in the patient. In addition, an effective amount of the autoimmune inhibitor in an immunosorbent column over which the patient's fluid is passed, is that amount which removes, neutralizes or inhibits the autoimmunogen(s) causing (directly or indirectly) or involved with the clinical manifestation(s) of the autoimmune disease in the patient. The effect of administering the autoimmune inhibitor and/or of extracorporeally passing fluid from the patient over immunosorbent(s) comprising the autoimmune inhibitor in accordance with the method of the present invention, can be seen as a slowing, interruption, inhibition, neutralization or prevention of the adaptive immune response associated with the autoimmune disease, often displayed as an alleviation of clinical manifestations of the disease. For example, the immunosuppressive effect of administering an effective amount of antibody to gamma IFN to a patient in need of such treatment would be the inhibition or prevention of further expression of gamma IFN by the patient, which could be quantitatively determined in terms of reduced fluid activity level of one or more of the elevated cytokines, i.e., gamma IFN or TNF-alpha. The lowering of the cytokine activity level may be measured directly in the treated patient, or the reduction in cytokine activity level may be projected from clinical studies in which dose regimens useful in achieving such reduction are established.

An effective amount of autoimmune inhibitor can be readily determined by the use of known techniques and by observing results obtained under analogous circumstances. In determining the effective amount or dose, a number of factors are considered by the attending diagnostician, including, but not limited to: the species of mammal; its size, age, and general health; the specific disease involved; the degree of or involvement or the severity of the disease; the response of the individual patient; as well as for purposes of administration, the particular compound being administered; the mode of administration; the bioavailability characteristics of the preparation administered; the dose regimen selected; the use of concomitant medication; and other relevant circumstances.

The autoimmune inhibitor of the present invention may comprise a single compound or anti-cytokine, e.g., anti-gamma IFN antibody administered to the patient or used in extracorporeal immunosorption, or it may be a combination of anti-cytokines or compounds, e.g., a combination of antibodies to IFNs, TNFs, and the like, administered to the patient or used in extracorporeal immunosorption, and/or antigens such as a target cell, including a CD4 cell, used in extracorporeal immunosorption. When combined, the compounds may be used concomitantly in an admixture or as simultaneous processes, or the compounds may be used sequentially to provide a combined effect without being in physical combination. For example, an AIDS patient may be treated by passing his blood, plasma or the like extracorporeally over an immunosorbent comprising CD4 cells to remove autoimmune antibodies against his own CD4 cells, while at the same time, or sequentially, anti-cytokines may be administered to neutralize, for instance the interferons and TNFs that have been induced within his body. The sequential treatments may occur in any order, so long as the autoimmune inhibitors have the desired anti-autoimmune effect.

Combined treatments, comprising the use of one or more autoimmune inhibitors in accordance with a preferred embodiment of the invention, may be mechanistically advantageous. This is because although circulating immunogens can be removed extracorporeally by passing the patient's body fluid over an immunosorbent comprising the autoimmune inhibitor(s), the administration of suitable autoimmune inhibitor(s), such as anti-cytokine antibodies, can effectively neutralize the immunogens, such as cytokines, both in circulation and on the cell surface. For example, to remove autoantibodies to CD4 cells, CD4 cells must be placed into an immunosorbent column. The body fluid from the patient is extracorporeally exposed to an immunosorbent comprising CD4. cells or their fragments, then the treated fluid (minus the antibodies that would otherwise attack his own CD4 cells) is returned to the patient. An attending diagnostician, physician or other person skilled in the art, can readily identify those patients who are in need of administrative treatment with an autoimmune inhibitor, or those who would benefit from extracorporeal treatment of their body fluids, or those who would benefit from a combination of the two.

The compound(s) comprising the autoimmune inhibitor, e.g., antibodies to IFNs, TNFs, and the like, and/or antigens such as a target cell, including CD4 cells, in accordance with the methods of the present invention, include cytotoxic amino acid sequence and glycosylation variants which also are used herein. The terms likewise cover biologically active functional equivalents, derivatives, or allelic or species variants of each compound, e.g., those differing by one or more amino acids(s) in the overall sequence. Further, the terms used in this application are intended to cover substitution, deletion and insertion amino acid variants of each compound, or post-translational modifications thereof.

Removal, neutralization and/or inhibition of alpha and gamma IFNs, TNF, and HLA class II antigen, and the like, and/or their receptors can be accomplished by the administration to the patient of one or more antibodies, or by including one or more antibodies in the immunosorbent over which the patient's body fluid is passed for extracorporeal treatment. As used herein, the term “antibody” is intended to include monoclonal or polyclonal antibodies, or a combination thereof, humanized forms of the monoclonal antibodies (comprising only human antibody protein), and chimeric monoclonal antibodies, camelid and heavy chain antibodies, as well as biologically active fragments, functional equivalents, derivatives, or allelic or species variants thereof. Treatment can include polyclonal antibodies from different animal species.

The term “biologically active fragment” is intended to mean a part of the complete molecule which retains all or some of the catalytic or biological activity possessed by the complete molecule, especially activity that allows specific binding of the antibody to an antigenic determinant.

“Functional equivalents” of an antibody include any molecule capable of specifically binding to the same antigenic determinant as the antibody, thereby neutralizing the molecule, e.g., antibody-like molecules, such as single chain antigen binding molecules.

“Derivative” is intended to include both functional and chemical derivatives, including fragments, segments, variants or analogs of a molecule. A molecule is a “chemical derivative” of another, if it contains additional chemical moieties not normally a part of the molecule. Such moieties may improve the molecule's solubility, absorption, biological half life, and the like, or they may decrease toxicity of the molecule, eliminate or attenuate any undesirable side effect of the molecule, and the like. Moieties capable of mediating such effects are disclosed in Remington's Pharmaceutical Sciences (1980). Procedures for coupling such moieties to a molecule are well known in the art. For example, the antibody of the present invention may be PEGylated prior to administration to a patient. Polyethylene glycol (PEG) moieties are attached to the antibody by a covalent attachment.

A “variant” or “allelic or species variant” of a protein refers to a molecule substantially similar in structure and biological activity to the protein. Thus, if two molecules possess a common activity and may substitute for each other, it is intended that they are “variants,” even if the composition or secondary, tertiary, or quaternary structure of one of the molecules is not identical to that found in the other, or if the amino acid or nucleotide sequence is not identical.

The term “interferon or IFN” is intended to refer to any known subtype of IFN. For example, “alpha IFN” is broadly intended to include any of the known 15 subtypes of alpha IFN, or any that may be determined in the future. Gamma IFN is particularly important in the present invention. The term “HLA class II antigens” is intended to mean not only HLA class II antigens, but also where appropriate, HLA class I or III antigens.

Any animal (mouse, rabbit, human, camel, llama, etc.) which is known to produce antibodies can be utilized to produce antibodies with the desired specificity. Methods for immunization are well known in the art. Such methods include subcutaneous or interperitoneal injection of the polypeptide. One skilled in the art will recognize that the amount of polypeptide used for immunization will vary based on the animal which is immunized, the antigenicity of the polypeptide and the site of injection. Chimeric antibodies, generated by recognized methods can also be used, including antibodies produced by recombinant methods.

If the antibody is to be administered intramuscularly or intravenously into the patient, then it may be preferable to use a substantially purified monoclonal antibody produced in human hybridoma. Humanized forms of the antibodies of the present invention may be generated using one of the procedures known in the art such as chimerization or CDR grafting. Also monoclonal antibodies of completely human protein may be applied. Until a satisfactory partner for human B-cells or activated human B-cells suitable for fusion become more readily available, a recognized procedure based upon immortalization of human B-cells with Epstein-Barr virus has provided as a source of human antibodies (see, Burton, Hospital Practice (August 1992), 67-74).

The antibodies useful in the methods of the present invention may be polyclonal antibodies, monoclonal antibodies, synthetic antibodies such as a biologically active fragment of the antibody, or they may be humanized monoclonal antibodies. Methods of making and using each of the types of antibodies useful in the methods of the invention are now described.

When the antibody used in the methods of the invention is a polyclonal antibody (IgG), the antibody is generated by inoculating a suitable animal with the autoimmune inhibitor of interest or a fragment thereof. Antibodies produced in the inoculated animal which specifically bind the autoimmune inhibitor of interest are then isolated from fluid obtained from the animal. Antibodies may be generated in this manner in several non-human mammals such as, but not limited to goat, sheep, horse, rabbit, and donkey. Methods for generating polyclonal antibodies are well known in the art and are described, for example in Harlow, et al. (1988, In: Antibodies, A Laboratory Manual, Cold Spring Harbor, N.Y.). These methods are not repeated herein as they are commonly used in the art of antibody technology.

When the antibody used in the methods of the invention is a monoclonal antibody, the antibody is generated using any well known monoclonal antibody preparation procedures such as those described, for example, in Harlow et al. (supra) and in Tuszynski et al. (1988, Blood, 72:109-115). In general, techniques for preparing monoclonal antibodies are well known in the art (Campbell, A. M., “Monoclonal Antibody Technology: Laboratory Techniques in Biochemistry and Molecular Biology,” Elsevier Science Publishers, Amsterdam, The Netherlands (1984); St. Groth et al., J. Immunol Methods 35:1-21 (1980). For example, in one embodiment an antibody capable of binding to gamma IFN is generated by immunizing an animal with natural, synthetic or recombinant gamma IFN. Given that these methods are well known in the art, they are not replicated herein. Generally, monoclonal antibodies directed against a desired antigen are generated from mice immunized with the antigen using standard procedures as referenced herein. Monoclonal antibodies directed against full length or peptide fragments of the autoimmune inhibitor of interest may be prepared using the techniques described in Harlow, et al. (supra).

When the antibody used in the methods of the invention is a biologically active antibody fragment or a synthetic antibody corresponding the antibody, the antibody is prepared as follows: a nucleic acid encoding the desired antibody or fragment thereof is cloned into a suitable vector. The vector is transfected into cells suitable for the generation of large quantities of the antibody or fragment thereof. DNA encoding the desired antibody is then expressed in the cell thereby producing the antibody. The nucleic acid encoding the desired peptide may be cloned and sequenced using technology which is available in the art, and described, for example, in Wright et al. (1992, Critical Rev. in Immunol. 12(3,4):125-168) and the references cited therein. Alternatively, quantities of the desired antibody or fragment thereof may also be synthesized using chemical synthesis technology. If the amino acid sequence of the antibody is known, the desired antibody can be chemically synthesized using methods known in the art.

The present invention also includes the use of humanized antibodies specifically reactive with epitopes of the autoimmune inhibitor of interest. These antibodies are capable of neutralizing the human form of the autoimmune inhibitor of interest. The humanized antibodies of the invention have a human framework and have one or more complementarity determining regions (CDRs) from an antibody, typically a mouse antibody, specifically reactive with the autoimmune inhibitor of interest. Thus, for example, humanized antibodies to gamma interferon are useful in the treatment of skin-related autoimmune diseases such as alopecia areata, dystrophic epidermolysis bullosa, vitiligo, and psoriasis, as well as graft-versus-host disease, rejection of transplant tissue, particularly bone marrow, and other autoimmune diseases, including SLE, AIDS, RA, diabetes, and the diseases listed in Table 1. Humanized antibody to gamma IFN is exemplified in Vasquez, et al., (U.S. Pat. No. 6,329,511). Further humanized antibodies to TNF-alpha are useful in the treatment of psychological diseases such as schizophrenia.

When the antibody used in the invention is humanized, the antibody may be generated as described in Queen, et al. (U.S. Pat. No. 6,180,370), Wright et al., (supra) and in the references cited therein, or in Gu et al. (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as human gamma IFN, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

Human constant region (CDR) DNA sequences from a variety of human cells can be isolated in accordance with well known procedures. Preferably, the human constant region DNA sequences are isolated from immortalized B-cells as described in WO87/02671, which is herein incorporated by reference. CDRs useful in producing the antibodies of the present invention may be similarly derived from DNA encoding monoclonal antibodies capable of binding to the autoimmune inhibitor of interest. Such humanized antibodies may be generated using well known methods in any convenient mammalian source capable of producing antibodies, including, but not limited to, mice, rats, rabbits, or other vertebrates. Suitable cells for constant region and framework DNA sequences and host cells in which the antibodies are expressed and secreted, can be obtained from a number of sources such as the American Type Culture Collection, Manassas, Va.

In addition to the humanized antibodies discussed above, other “substantially homologous” modifications to native antibody sequences can be readily designed and manufactured utilizing various recombinant DNA techniques well known to those skilled in the art. Moreover, a variety of different human framework regions may be used singly or in combination as a basis for humanizing antibodies directed to the autoimmune inhibitor of interest. In general, modifications of genes may be readily accomplished using a variety of well-known techniques, such as site-directed mutagenesis (Gillman and Smith, Gene, 8:81-97 (1979); Roberts et al., 1987, Nature, 328:731-734).

Substantially homologous sequences to antibody sequences of the autoimmune inhibitor of interest are those which exhibit at least about 85% homology, usually at least about 90%, and preferably at least about 95% homology with a reference immunoglobulin protein. For example, a substantially homologous sequence to antibody to gamma IFN are those which exhibit at least about 85% homology, usually at least about 90% homology, and preferably at least about 95% homology with a reference gamma IFN immunoglobulin protein.

Alternatively, polypeptide fragments comprising only a portion of the primary antibody structure may be produced, which fragments possess one or more functions of the antibody to the autoimmune inhibitor of interest, for example, gamma IFN antibody. These polypeptide fragments may be generated by proteolytic cleavage of intact antibodies using methods well known in the art, or they may be generated by inserting stop codons at the desired locations in vectors comprising the fragment using site-directed mutagenesis.

DNA encoding antibody to the autoimmune inhibitor of interest is expressed in a host cell driven by a suitable promoter regulatory sequence which is operably linked to the DNA encoding the antibody. Typically, DNA encoding the antibody is cloned into a suitable expression vector such that the sequence encoding the antibody is operably linked to the promoter/regulatory sequence. Such expression vectors are typically replication competent in a host organism either as an episome or as an integral part of the host chromosomal DNA. Commonly, an expression vector will comprise DNA encoding a detectable marker protein, e.g., a gene encoding resistance to tetracycline or neomycin, to permit detection of cells transformed with the desired DNA sequences (U.S. Pat. No. 4,704,362).

E. coli is an example of a prokaryotic host which is particularly useful for expression of DNA sequences encoding the antibodies of the present invention. Other microbial hosts suitable for use include but are not limited to, Bacillus subtilis, and other enterobacteriaceae, such as Salmonella, Serratia, and various Pseudomonas species. It is possible to generate expression vectors suitable for the desired host cell wherein the vectors will typically comprise an expression control sequence which is compatible with the host cell. A variety of promoter/regulatory sequences are useful for expression of genes in these cells, including but not limited to the lactose promoter system, a tryptophan (trp) promoter system, a beta-lactamase promoter system, or a promoter system derived from phage lambda. The promoter will typically control expression of the antibody in which the DNA sequence is operably linked thereto, the promoter is optionally linked with an operator sequence and generally comprises RNA polymerase and ribosome binding site sequences and the like for initiating and completing transcription and translation of the desired antibody.

Yeast is an example of a eukaryotic host useful for cloning DNA sequences encoding the antibodies of the present invention. Saccharomyces is a preferred eukaryotic host. Promoter/regulatory sequences which drive expression of nucleic acids in eukaryotic cells include but are not limited to the 3-phosphoglycerate kinase promoter/regulatory sequence and promoter/regulatory sequences which drive expression of nucleic acid encoding other glycolytic enzymes.

In addition to microorganisms, mammalian tissue cell culture may also be used to express and produce the antibodies of the present invention (Winnacker, 1987, “From Genes to Clones,” VCH Publishers, New York, N.Y.). Eukaryotic cells are preferred for expression of antibodies and a number of suitable host cell lines have been developed in the art, including Chinese Hamster Ovary (CHO) cells, various COS cell lines, HeLa cells, preferably myeloma cell lines, and transformed B-cells or hybridomas. Expression vectors which express desired sequences in these cells can include expression control sequences, such as an origin of DNA replication, a promoter, an enhancer (Queen et al., 1986, Immunol. Rev., 89, 49-68), and necessary processing sequence sites, such as ribosome binding sites, RNA splice sites, polyadenylation sites, and transcriptional initiation and terminator sequences. Preferred expression control sequences are promoters derived from immunoglobulin genes, SV40, adenovirus, cytomegalovirus, bovine papilloma virus and the like.

The vectors containing the DNA segments of interest can be transferred into the host cell by well-known methods, which vary depending on the type of cellular host. For example, calcium chloride transfection is commonly utilized for prokaryotic cells, whereas calcium phosphate treatment or electroporation may be used for other cellular hosts. (Sambrook et al., 1989, Molecular Cloning, A Laboratory Manual, Cold Spring Harbor, N.Y.).

One of skill in the art will further appreciate that the present invention encompasses the use of antibodies derived from camelid species. That is, the present invention includes, but is not limited to, the use of antibodies derived from species of the camelid family. As is well known in the art, camelid antibodies differ from those of most other mammals in that they lack a light chain, and thus comprise only heavy chains with complete and diverse antigen binding capabilities (Hamers-Casterman et al., 1993, Nature, 363:446-448). Such heavy-chain antibodies are useful in that they are smaller than conventional mammalian antibodies, they are more soluble than conventional antibodies, and further demonstrate an increased stability compared to some other antibodies.

Camelid species include, but are not limited to Old World camelids, such as two-humped camels (C. bactrianus) and one humped camels (C. dromedarius). The camelid family further comprises New World camelids including, but not limited to llamas, alpacas, vicuna and guanaco. The use of Old World and New World camelids for the production of antibodies is contemplated in the present invention, as are other methods for the production of camelid antibodies set forth herein.

The production of polyclonal sera from camelid species is substantively similar to the production of polyclonal sera from other animals such as sheep, donkeys, goats, horses, mice, chickens, rats, and the like. The skilled artisan, when equipped with the present disclosure and the methods detailed herein, can prepare high-titers of antibodies from a camelid species with no undue experimentation. As an example, the production of antibodies in mammals is detailed in such references as Harlow et al., (1989, Antibodies: A Laboratory Manual, Cold Spring Harbor, N.Y.). Camelid species for the production of antibodies and sundry other uses are available from various sources, including but not limited to, Camello Fataga S. L. (Gran Canaria, Canary Islands) for Old World camelids, and High Acres Llamas (Fredricksburg, Tex.) for New World camelids.

The isolation of camelid antibodies from the serum of a camelid species can be performed by many methods well known in the art, including but not limited to ammonium sulfate precipitation, antigen affinity purification, Protein A and Protein G purification, and the like. As an example, a camelid species may be immunized to a desired antigen, for example an interferon gamma, IL-1, or tumor necrosis factor alpha peptide, or fragment thereof, using techniques well known in the art. The whole blood can them be drawn from the camelid and sera can be separated using standard techniques. The sera can then be absorbed onto a Protein G-Sepharose column (Pharmacia, Piscataway, N.J.) and washed with appropriate buffers, for example 20 mM phosphate buffer (pH 7.0). The camelid antibody can then be eluted using a variety of techniques well known in the art, for example 0.15 M NaCl, 0.58% acetic acid (pH 3.5). The efficiency of the elution and purification of the camelid antibody can be determined by various methods, including SDS-PAGE, Bradford Assays, and the like. The fraction that is not absorbed can be bound to a Protein A-Sepharose column (Pharmacia, Piscataway, N.J.) and eluted using, for example 0.15 M NaCl, 0.58% acetic acid (pH 4.5). The skilled artisan will readily understand that the above methods for the isolation and purification of camelid antibodies are exemplary, and other methods for protein isolation are well known in the art and are encompassed in the present invention.

The present invention further contemplates the production of camelid antibodies expressed from nucleic acid. Such methods are well known in the art, and are detailed in, for example U.S. Pat. Nos. 5,800,988; 5,759,808; 5,840,526, and 6,015,695, which are incorporated herein by reference in their entirety. Briefly, cDNA can be synthetised from camelid spleen mRNA. Isolation of RNA can be performed using multiple methods and compositions, including TRIZOL (Gibco/BRL, La Jolla, Calif.) further, total RNA can be isolated from tissues using the guanidium isothiocyanate method detailed in, for example, Sambrook et al. (1989, Molecular Cloning, A Laboratory Manual, Cold Spring Harbor, N.Y.). Methods for purification of mRNA from total cellular or tissue RNA are well known in the art, and include, for example, oligo-T paramagnetic beads. cDNA synthesis can then be obtained from mRNA using mRNA template, an oligo dT primer and a reverse transcriptase enzyme, available commercially from a variety of sources, including Invitrogen (La Jolla, Calif.). Second strand cDNA can be obtained from mRNA using RNAse H and E. coli DNA polymerase I according to techniques well known in the art.

Identification of cDNA sequences of relevance can be performed by hybridization techniques well known by one of ordinary skill in the art, and include methods such as Southern blotting, RNA protection assays, and the like. Probes to identify variable heavy immunoglobulin chains (V_{HH}) are available commercially and are well known in the art, as detailed in, for example, Sastry et al., (1989, Proc. Nat'l. Acad. Sci. USA, 86:5728). Full-length clones can be produced from cDNA sequences using any techniques well known in the art and detailed in, for example, Sambrook et al. (1989, Molecular Cloning, A Laboratory Manual, Cold Spring Harbor, N.Y.).

The clones can be expressed in any type of expression vector known to the skilled artisan. Further, various expression systems can be used to express the V_{HH} peptides of the present invention, and include, but are not limited to eukaryotic and prokaryotic systems, including bacterial cells, mammalian cells, insect cells, yeast cells, and the like. Such methods for the expression of a protein are well known in the art and are detailed elsewhere herein.

The V_{HH} immunoglobulin proteins isolated from a camelid species or expressed from nucleic acids encoding such proteins can be used directly in the methods of the present invention, or can be further isolated and/or purified using methods disclosed elsewhere herein.

The present invention is not limited to V_{HH} proteins isolated from camelid species, but also includes V_{HH} proteins isolated from other sources such as animals with heavy chain disease (Seligmann et al., 1979, Immunological Rev. 48:145-167, incorporated herein by reference in its entirety). The present invention further comprises variable heavy chain immunoglobulins produced from mice and other mammals, as detailed in Ward et al. (1989, Nature 341:544-546, incorporated herein by reference in its entirety). Briefly, V_{H} genes were isolated from mouse splenic preparations and expressed in E. coli. The present invention encompasses the use of such heavy chain immunoglobulins in the treatment of various autoimmune disorders detailed herein.

As used herein, the term “heavy chain antibody” or “heavy chain antibodies” comprises immunoglobulin molecules derived from camelid species, either by immunization with an peptide and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

Once expressed, whole antibodies, dimers derived therefrom, individual light and heavy chains, or other forms of antibodies can be purified according to standard procedures known in the art. Such procedures include, but are not limited to, ammonium sulfate precipitation, the use of affinity columns, routine column chromatography, gel electrophoresis, and the like (see, generally, R. Scopes, “Protein Purification”, Springer-Verlag, N.Y. (1982)). Substantially pure antibodies of at least about 90% to 95% homogeneity are preferred, and antibodies having 98% to 99% or more homogeneity most preferred for pharmaceutical uses. Once purified, the antibodies may then be used therapeutically.

The autoimmune inhibitor antibody(ies) also may be produced and/or isolated from discordant animal species. For example, porcine or bovine antibodies may be used for the treatment of humans. To use animal-derived antibodies for a prolonged period, antibodies from a variety of different animal species must be used, permitting the source of the antibodies to be changed if the patient develops a hypersensitivity or deleterious response to a component of the originally administered antibody, antibody fragment or polypeptide. In some cases, to prevent allergenic reaction between injections of antibodies from a discordant species, immunodepressant drugs, such as steroid hormones or cyclophosphamide are administered. A preferred compound of the present invention is derived from a mature compound from recombinant microbial cell culture, prepared, isolated and substantially purified in accordance with known techniques. A combination of monoclonal and polyclonal antibodies can also be utilized.

To evaluate the antibody or antibodies, conditions for incubating the antibody or antibodies with a test sample vary. Incubating conditions depend on the format employed in the assay, the detection methods employed, the nature of the test sample, and the type and nature of the antibody used in the assay. One skilled in the art will recognize that any one of the commonly available immunological assay formats (such as radioimmunoassays, enzyme-linked immunosorbent assays, diffusion based Ouchterlony, or rocket immunofluorescent assays, or the like) can readily be adapted to employ the antibodies of the present invention.

Autoimmune inhibitor(s) of the present invention include polypeptides comprising the epitope of the antibody or biologically active fragment thereof, or polypeptide that is functional in conferring protection in the individual suffering from autoimmune disease, or functionally conserved fragments or amino acid variants thereof. Identification of the epitope is a matter of routine experimentation. Most typically, one would conduct systematic substitutional mutagenesis of the compound molecule while observing for reductions or elimination of cytoprotective or neutralizing activity. In any case, it will be appreciated that due to the size of many of the antibodies, most substitutions will have little effect on binding activity. The great majority of variants will possess at least some cytoprotective or neutralizing activity, particularly if the substitution is conservative. Conservative amino acid substitutions are substitutions from the same class, defined as acidic (Asp, Glu), hydroxy-like (Cys, Ser, Thr), amides (Asn, Gln), basic (His, Lys, Arg), aliphatic-like (Met, Ile, Leu, Val, Gly, Ala, Pro), and aromatic (Phe, Tyr, Trp).

Homologous antibody or polypeptide sequences generally will be greater than about 30 percent homologous on an identical amino acid basis, ignoring for that purposes of determining homology any insertions or deletions from the selected molecule in relation to its native sequence. The compounds discussed herein, i.e., autoimmune inhibitors for administration to the patient with autoimmune disease and/or for removal, neutralization or inhibition of the autoimmunogen(s) by extracorporeal immunosorption in accordance with the present invention, also include glycosylation variants as well as unglycosylated forms of the agents, fusions of the agents with heterologous polypeptides, and biologically active fragments of the agents, again so long as the variants possess the requisite neutralizing or cytoprotective activity.

The autoimmune inhibitor antibody(ies) is also effective when immobilized on a solid support. Examples of such solid supports include, but are not limited to, plastics such as polycarbonate, complex carbohydrates such as agarose and sepharose, and acrylic resins, such as polyacrylamide and latex beads. Techniques for coupling antibodies to such solid supports are well known in the art (Weir et. al. “Handbook of Experimental Immunology” 4th Ed., Blackwell Scientific Publications, Oxford, England, Chap. 10 (1986), Jacoby et al., Meth. Enzym. 34 Academic Press, N.Y. (1974).

Additionally, one or more of the antibodies used in the above described methods can be detectably labeled prior to use. Antibodies can be detectably labeled through the use of radioisotopes, affinity labels (such as, biotin, avidin, etc.), enzymatic labels (such as horseradish peroxidase, alkaline phosphatase, etc.) fluorescent labels (such as, FITC or rhodamine, etc.), paramagnetic atoms, etc. Procedures for accomplishing such labeling are well-known in the art, for example see Stemberger et al., J. Histochem. Cytochem. 18:315 (1970); Bayer et al., Meth. Enzym. 62:308 (1979); Engval et al., Immunol 109:129 (1972); Goding, J. Immunol Meth. 13:215 (1976). The labeled antibodies of the present invention can be used for in vitro, in vivo, and in situ assays to identify cells or tissues which express a specific cytokine or antigenic protein.

For administration purposes, an effective amount of an autoimmune inhibitor is expected to vary from about 0.1 milligram per kilogram of body weight per day (mg/kg/day) to about 500 mg/kg/day, preferably from about 0.5 milligram per kilogram of body weight per day (mg/kg/day) to about 100 mg/kg/day, even more preferably from about 1 to about 50 mg/kg/day. Antibodies can be administered once a month, once every two weeks, once a week, several times a week, several times per day or once a day. Preferably, the composition is administered from about one to to about ten times per day, more preferably from about one to about five times a day, and more preferably, the composition is administered from one to three times per day. Most preferred is administration of the composition three times per day. Administration can continue for several days to several weeks to several months to about a year.

daily for several months or for about a month, or for one or more weeks, depending on need. If antibodies are used from a variety of species, a different antibody can be given every 5-6 days.

Cytokines and other pathological agents can also be neutralized or removed from the patient in accordance with the methods of the present invention by administering vaccines against the cytokines or agents. However, vaccines may be dangerous to use in vivo, unless the antibodies that may be induced by the treatment can be controlled. Otherwise, such vaccines, although initially effective, may lead to immunological disaster in the patient.

In effecting treatment of a patient, an autoimmune inhibitor can be administered in any form or mode which makes the compound bioavailable in effective amounts, including oral and parenteral routes. For example, autoimmune inhibitors can be administered by inhalation, orally, subcutaneously, intramuscularly, intravenously, transdermally, intranasally, rectally, and the like. Parenteral administration is generally preferred.

In particular, if the autoimmune inhibitor is an antibody, preferred routes of administration include intramuscular, intravenous, cutaneous, local, ionophoretic, inhalation, or as an ointment. One skilled in the art of preparing formulations can readily select the proper form and mode of administration depending upon the particular characteristics of the compound selected, the disease state to be treated, the stage of the disease, and other relevant circumstances.

The autoimmune inhibitor can be administered alone, or in the form of a pharmaceutical composition in combination with pharmaceutically acceptable carriers or excipients, the proportion and nature of which are determined by the solubility and chemical properties of the compound selected, the chosen route of administration, and standard pharmaceutical practice. The compounds of the invention, while effective themselves, may be formulated and administered in the form of their pharmaceutically acceptable acid addition salts for purposes of stability, convenience of crystallization, increased solubility and the like.

In one embodiment, the present invention provides a method of treatment in which the autoimmune inhibitor is admixed or otherwise associated with one or more inert carriers. These compositions are useful, for example, as assay standards, as convenient means of making bulk shipments, or as pharmaceutical compositions. An assayable amount of an autoimmune inhibitor is an amount which is readily measurable by standard assay procedures and techniques as are well known and appreciated by those skilled in the art. Assayable amounts of the autoimmune inhibitor will generally vary from about 0.001% to about 75% of the composition by weight. Inert carriers can be any material which does not degrade or otherwise covalently react with an autoimmune inhibitor. Examples of suitable inert carriers include water; aqueous buffers, such as those which are generally useful in High Performance Liquid Chromatography (HPLC) analysis; organic solvents, such as acetonitrile, ethyl acetate, hexane and the like; and pharmaceutically acceptable carriers or excipients.

More particularly, in accordance with the present invention, pharmaceutical compositions are provided comprising an effective amount of autoimmune inhibitor in admixture or otherwise in association with one or more pharmaceutically acceptable carriers or excipients.

The pharmaceutical compositions are prepared in a manner well known in the pharmaceutical art. The carrier or excipient may be a solid, semi-solid, or liquid material which can serve as a vehicle or medium for the active ingredient. Suitable carriers or excipients are well known in the art. The pharmaceutical composition may be adapted for oral, parenteral, or topical use, and may be administered to the patient in the form of tablets, powders, granules, capsules, suppositories, solution, suspensions, or the like.

The compounds of the present invention may be administered orally, for example, with an inert diluent or with an edible carrier. They may be enclosed in gelatin capsules or compressed into tablets. For the purpose of oral therapeutic administration, the compounds may be incorporated with excipients and used in the form of tablets, troches, capsules, elixirs, suspensions, syrups, wafers, chewing gums and the like. These preparations should contain a measurable amount of autoimmune inhibitor as the active ingredient, but the amount may vary depending upon the particular form and may conveniently be between about 1% to about 90% of the weight of the pharmaceutical composition. The amount of the compound present in compositions is such that a suitable dosage will be obtained. Preferred compositions and preparations according to the present invention are prepared so that an oral dosage unit form contains between 5.0 to 300 milligrams of an autoimmune inhibitor of the invention. Dosage, in tablet or capsule form, is at a preferred dose of 1 to 25 mg/kg patient body weight per day. The dose may be increased or decreased appropriately depending on the response of the patient, and patient tolerance.

The tablets, pills, capsules, troches and the like may also contain one or more of the following adjuvants: binders such as microcrystalline cellulose, starch paste, gum tragacanth or gelatin; excipients such as starch or lactose, disintegrating agents such as alginic acid, corn starch and the like; lubricants such as magnesium stearate; glidants such as colloidal silicon dioxide; and sweetening agents such as sucrose or saccharin may be added, or a flavoring agent such as peppermint, methyl salicylate or orange flavoring, of the types usually used in the manufacture of medical preparations. When the dosage unit form is a capsule, it may contain, in addition to materials of the above type, a liquid carrier such as polyethylene glycol or a fatty oil. Other dosage uniforms may contain other various materials which modify the physical form of the dosage unit, for example, as coatings. Thus, tablets or pills may be coated with sugar, shellac, or other enteric coating agents.

For use in oral liquid preparation, the compound(s) may be prepared as a liquid suspension, emulsion, or syrup, being supplied either in liquid form or a dried form suitable for hydration in water or normal saline. A syrup may contain, in addition to the present compounds, sucrose as a sweetening agent and certain preservatives, dyes and colorings and flavors.

Materials used in preparing these various compositions should be pharmaceutically pure and non-toxic in the amounts used. As used herein, a protein is said to be “pharmaceutically pure” if the autoimmune inhibitor comprises no substance that would be harmful to the patient. A “substantially pure” or “substantially purified” protein is one in which specific activity cannot be significantly increased by further purification, and if the specific activity is greater than that found in whole cell extracts containing the protein.

The method of the present invention is also accomplished by injecting the selected compound(s) in the autoimmune inhibitor, e.g., intravenously, intramuscularly, intradermally, or subcutaneously, in the form of aqueous solutions, suspensions or oily or aqueous emulsions, such as liposome suspensions. Typically, for parenteral administration, the extract is formulated as a lipid, e.g., triglyceride, or phospholipid suspension, with the extract components being dissolved in the lipid phase of the suspension. These preparations should contain at least 0.1% of an autoimmune inhibitor of the invention, but may be varied to be between 0.1 and about 50% of the weight thereof. The amount of autoimmune inhibitor present in such compositions is such that a suitable dosage will be obtained. Preferred compositions and preparations according to the present invention are prepared so that a parenteral dosage unit contains between 5.0 to 100 milligrams of autoimmune inhibitor. Dosage level may be increased or decreased appropriately, depending on the conditions of disease, the age of the patient, etc.

If the autoimmune inhibitor is an antibody, the antibody is administered to a patient in an amount effective to treat the condition. The effective amount for treatment depends upon the severity of the condition and the general state of the patient's own immune system, but generally the amount ranges from about 0.01 to about 100 milligrams of antibody per dose, with dosages from 0.1 to 50 milligrams and 1 to 10 milligrams per patient being more commonly used. Single or multiple administrations on a daily, weekly or monthly schedule can be carried out with dose levels and pattern being selected by the treating physician.

The solutions or suspensions may also include one or more of the following adjuvants: sterile diluents such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as ethylene diaminetetraacetic acid; buffers such as acetates, citrates or phosphates and agents for the adjustment of tonicity such as sodium chloride or dextrose.

The parenteral preparation can be enclosed in ampules, disposable syringes or multiple dose vials made of glass or plastic.

Moreover, the invention provides for the treatment of a patient with autoimmune disease by the use (administration or use in extracorporeal immunosorbent) of one or more antisense molecules, which are characterized by the ability to bind to the autoimmunogen, or a functionally equivalent derivative, or allelic or species variant thereof.

“Antisense sequence,” or “antisense molecule” refers to peptides derived from pseudogenes which are constructed by reversing the orientation of the gene encoding the autoimmunogen with regard to its promoter, so that the antisense strand is transcribed. The term also refers to the antisense strand of RNA or of cDNA which compliments the strand of DNA encoding the cytokine, autoimmunogen, protein or peptide of interest.

When introduced into the patient, the antisense molecule binds to, neutralizes or inhibits the autoimmunogen, much the same as an antibody. Thus, the present methods can be practiced by means of one or more antisense molecules. Moreover, when the nucleic acid sequence encoding the autoimmune anti-sense molecule is introduced into the cells under the control of a promoter, the anti-sense gene molecule binds to, neutralizes or inhibits the gene(s) encoding the autoimmunogen(s), inhibiting or preventing further pathogenesis. The inhibition appears to depend on the formation of an RNA-RNA or cDNA-RNA duplex in the nucleus or in the cytoplasm. Thus, if the antisense gene is stably introduced into a cultured cell, the normal processing and/or transport is affected if a sense-antisense duplex forms in the nucleus; or if antisense RNA is introduced into the cytoplasm of the cell, the expression or translation of the autoimmunogen is inhibited. Such antisense nucleic acid sequences may further include modifications which could affect the biological activity of the antisense molecule, or its manner or rate of expression. Such modifications may also include, e.g., mutations, insertions, deletions, or substitutions of one or more nucleotides that do not affect the function of the antisense molecule, but which may affect intracellular localization. Also, the nucleic acid sequence may determine an uninterrupted antisense RNA sequence or it may include one or more introns.

In a particular embodiment of the invention, a unique combination of compounds may be combined to form the autoimmune inhibitor to be used for the treatment of multiple sclerosis (“MS”), for which there is no other rational treatment. The administration of beta interferon has been shown to decrease the rate of exacerbation of the disease in some patients. This positive effect can be explained by the fact that beta IFN decreases the synthesis of gamma IFN and TNF (Henniger et al., Neurology 46:1633-1639 (1996)). These data both confirm the negative effect of gamma IFN and TNF on the autoimmune process, and validate the synergic action in MS of anti-cytokine antibodies (anti-gamma IFN antibodies and anti-TNF antibodies) together with the administration of the cytokine beta IFN to decrease the production of gamma IFN and TNF. MS may also be treatable using antibodies to gamma IFN alone.

In one embodiment of the invention, treatment comprises passing the fluid drawn from the patient over immunosorbent comprising the autoimmune inhibitor, followed by returning the treated fluid to its source. This method is particularly suited for treating certain autoimmune conditions in which the autoimmune inhibitor cannot be administered to the patient. For example, in a preferred embodiment, the patient's fluid is exposed to an immunosorbent comprising an effective amount of target cells, CD4 cells, and/or DNA, to remove, neutralize or inhibit the autoantibodies in the patient's fluid, followed by returning the treated fluid to the patient. The immunosorbent for extracorporeal treatment may further comprise one or more antibodies (e.g., anti-alpha IFN antibodies, antibodies to alpha IFN receptor, anti-gamma IFN antibodies, antibodies to gamma IFN receptor, anti-TNF antibodies, antibodies to TNF receptor, antibodies to an HLA class II antigen or to its receptor, or immunoglobulin E (“IgE”).

To counter transplant rejection, antibodies to alpha IFN and gamma IFN, or in some cases gamma IFN alone, and the antigen of the transplanted cell or organ are placed in the immunosorbent column. To treat myocardial infarction or stroke, antibodies to IFNs and cardiac or brain antigens, respectively, are placed in the immunosorbent column. Further, the present invention may he used in combination with immunosuppressive therapy to achieve the desired results.

In another preferred embodiment of the invention, the patient's fluid is extracorporeally exposed to an immunosorbent comprising target cells. For example, for the treatment of rheumatoid arthritis, target cell antigens from joints, skin, collagen, and possibly other target antigens, are used as immunosorbents, alone or in conjunction with other autoimmune inhibitors, such as antibodies to IFNs and/or TNF or their receptors; In addition, for the treatment of rheumatic fever, the invention provides an immunosorbent comprising antibodies to IFNs and/or TNF or their receptors and/or other substances, in conjunction with a second cardiac tissue sorbent for removing C autoantibodies against cardiac tissue. The second sorbent can also include selected serotypes of Streptococcus (group “A”), because certain antigens from cardiac tissue and some serotypes of Streptococcus are antigenically similar. For the treatment of autoimmune diseases of the central nervous system, target cell antigens from brain cells, e.g., to nuclear, membrane or cytoplasm antigens, are used to absorb autoantibodies formed against the brain cells.

In yet another preferred embodiment of the invention, the patient's fluid is extracorporeally exposed to an immunosorbent comprising DNA. For example, for the treatment of SLE the immunosorbent comprises DNA to remove, reduce or neutralize the patient's anti-DNA autoantibodies. For a description of anti-DNA antibodies as they appear in SLE, see Graninger et. al., J. Rheumatol. 18:1621-1622 (1981).

In a further preferred embodiment the fluid is extracorporeally exposed to an immunosorbent comprising antibody to IgE. For example, for treating certain diseases related to hypersensitivity of the immediate type, e.g., bronchial asthma, antibody to IgE is used as an immunosorbent, alone or in conjunction with other autoimmune inhibitors, such as antibodies to IFNs and/or TNF or their receptors.

In an additional preferred embodiment of the invention the patient's fluid is extracorporeally exposed to an immunosorbent comprising CD4 cells. For example, for the treatment of AIDS, the immunosorbent comprises CD4 cells, alone or in conjunction with other autoimmune inhibitors, such as antibodies to IFNs and/or TNF and/or HLA class II antigen, or their receptors. The CD4 component of the immunosorbent comprises lymphocytes, primarily CD4 cells, from healthy donors to absorb serum autoantibodies which react with the patient's own CD4 cells.

For extracorporeal treatment, the pathogenic antibodies and/or immune lymphocytes can be removed or reduced by passing any of the previously described fluids over the prepared immunosorbent column comprising an autoimmune inhibitor. When using whole blood, plasma, or plasma with leukocytes, one can use a blood cell separator (e.g., Cobe “Spectra”) to which the immunosorbent column is connected. See, e.g., U.S. Pat. No. 4,362,155, which is incorporated herein by reference. To remove pathological substances from joint or spinal fluids or the like, a special extracorporeal device with a small amount of immunosorbent is used. To neutralize antibodies to autoimmunogens, such as antibodies to target cells, including CD4 cells, the cells themselves or that portion of the cells containing the antigenic determinant(s) for the subject antibodies, must be placed directly in the immunosorbent column.

For the removal of compound(s) by extracorporeal immunosorption in accordance with the present invention, particles of sorbent material, such as amorphous silica or Sepharose, can be readily placed in a container to prepare the immunosorbent for the extracorporeal procedure. The container can be constructed of any material which can readily undergo steam, chemical, or gamma-irradiation sterilization. For instance, glass, polycarbonate, polystyrene, polymethylmethacrylate, polyolefins such as polyethylene and polypropylene, are all suitable.

Various ways of retaining or immobilizing sorbent material within a container are available. For instance, sorbent material may be placed between layers of retaining filters, or placed within a porous solid matrix. The solid matrix immobilizes the sorbent, while simultaneously permitting flow of blood or other fluids, and contact with the sorbent. As is readily apparent to one of ordinary skill in the art, a wide variety of structures arc available for providing suitable fluid/sorbent contact, structures which do not cause significant hemolysis. Prudent use of additional filters to retain the sorbent particles in their container is preferred. The pretreated, immobilized sorbent may be contacted with the fluid in a variety of ways, e.g., admixture, elution, and the like, which would be recognized in the art.

Although a columnar sorbent bed is exemplified in Example 1, beds of any other shape capable of functioning in the manner described herein may also be used. The length-to-diameter ratio of the sorbent bed should be selected so as to minimize any pressure drop along the bed, and to ensure that shear rates remain below the known values that correlate with cellular damage or destruction. The pressure drop along the sorbent bed (and thus the increase in shear rate) is directly proportional to the length of the bed. However, mitigating against use of a short bed is the fact that clearance of a substance from the fluid increases with a longer bed. The capability of the sorbent to adsorb can be assessed by experiments in which a test solution (such as whole blood or plasma) is contacted with the prepared sorbent at a constant temperature. The data generated from such an experiment can be used to determine an equilibrium constant (K), according to which the capacity of the prepared sorbent is determined. An equilibrium constant (K) is defined in units of (ml solution/g composition). The capacity of a composition provides a way to estimate the mass of the prepared sorbent required to remove a certain quantity of material, such as a cytokine, from solution.

In one embodiment of the invention, one skilled in the art will readily recognize that the disclosed autoimmune inhibitor or immunosorbent comprising the autoimmune inhibitor of the present invention can readily be incorporated into one of the established kit formats which are well known in the art. While in yet another embodiment of the present invention, kits are provided which contain the necessary reagents to carry out the previously described methods. For example, in one instance such a kit comprises a pharmaceutical composition or antibody cocktail comprising the necessary autoimmune inhibitor, with or without pharmaceutically acceptable carriers, excipients and the like, in an amount suitable for administration to a patient suffering from an autoimmune disease. In another instance, such a kit comprises the autoimmune inhibitor bound to an immunosorbent that may be used for the extracorporeal treatment of autoimmune disease in a patient. In particular, such a kit comprises an effective amount to extracorporeally remove, reduce or neutralize one or more autoimmunogens from the fluid of a patient with autoimmune disease of at least one of the following: anti-alpha IFN antibodies, antibodies to alpha IFN receptor, anti-gamma IFN antibodies, antibodies to gamma IFN receptor, anti-TNF antibodies, antibodies to TNF receptor, antibodies to an HLA class II antigen or to its receptor, and/or antibodies to IgE. Another preferred kit comprises an effective amount to extracorporeally remove, reduce or neutralize one or more autoantibodies from the fluid of a patient with autoimmune disease of at least one of the following: target cells, CD4 cells, or DNA. While, yet additional kits comprise components of each of the previously defined kits, to provide the combined treatments of the present invention.

The present invention further encompasses methods for treating psychological diseases. Such diseases include, but are not limited to, schizophrenia and the like. This is because, as demonstrated by the data disclosed elsewhere herein, levels of TNF-alpha in a schizophrenic patient correlate with a patient's well being and mental state. Further, the data disclosed herein demonstrate that administration of an effective amount of an antibody to TNF-alpha results in, among other things, an improvement in a patient's mood, energy, sociability, physical activity, a decrease in negative symptoms as measured by PANSS (Positive and Negative Syndrome Scale; the standard test for evaluating schizophrenics), and a decrease in motor retardation and depression. As further demonstrated by the data disclosed herein, the administration of antibodies to TNF-alpha results in the alleviation of other symptoms associated with or mediated by schizophrenia. Such symptoms include emotional withdrawal, passive apathetic social withdrawal, will disturbance, and active social withdrawal.

The method comprises administering an antibody to TNF-alpha to a patient with schizophrenia. The antibody is administered in an effective amount, as disclosed elsewhere herein. As an example, antibodies to TNF-alpha can be administered intramuscularly, intravenously, intradermally, cutaneously, ionophoretically, topically, locally, and by inhalation, preferably by parenteral administration. The concentration of anti-TNF-alpha antibodies can be from about 1 μg/ml to about 500 μg/ml, preferably from about 10 μg/ml to about 200 μg/ml, even more preferably from about 20 μg/ml to about 100 μg/ml, yet more preferably from about 30 μg/ml to about 75 μg/ml, preferably about 66 μg/ml. The amount of anti-TNF-alpha antibody administered to a patient can be from about 0.1 ml to about 10 ml, preferably from about 0.5 ml to about 7 ml, more preferably from about 1 ml to about 5 ml, even more preferably about 2 ml. The anti-TNF-alpha antibody can be administered from about once a year to about twice per year to several times a year to monthly to a few times a month to several times a month to weekly to several times a week to daily, to twice daily to several times a day. Preferably, the anti-TNF-alpha antibody is administered to a patient about twice daily for about five consecutive days. This process may be repeated, as can be determined by one of skill in the art.

Methods for recognizing and diagnosing schizophrenia are well known in the art and are described in, for example, The Diagnostic and Statistical Manual of Psychiatric Disorders (4th edition, 1994, American Psychiatric Association, Washington D.C.)

As discussed elsewhere herein, an antibody comprises a polyclonal antibody, a monoclonal antibody, a humanized antibody, a camelid or heavy chain antibody, and a synthetic antibody. The present invention further encompasses a biologically active fragment of an antibody, a functional equivalent of an antibody, a derivative of an antibody, an allelic variant of an antibody, and a species variant of an antibody. The antibodies, fragments, equivalents, derivatives, and variants thereof necessary to practice the methods of the present invention will be apparent to one of skill in the art when supplied with the present disclosure. The skilled artisan will further appreciate that the present invention is not limited to the singular administration of an antibody, fragment, equivalent, derivative, or variant thereof, but rather that they may be administered in a combination, either in combination with each other or in a temporal sense.

The method of the present invention further includes routes in which to administer an antibody to TNF-alpha to a patient. The skilled clinician will recognize that routes of administration may vary, depending on the status and needs of the patient, the resources available, the severity of the disease, and the like. However, as amply disclosed by the teachings provided herein, the route of administration can include, but is not limited to intramuscular, intravenous, intradermal, cutaneous, ionophoretical, topical, local, and inhalation administration. Thereby, the skilled artisan will be able to easily determine the best route of administration with little or no undue experimentation.

As a non-limiting example, a patient diagnosed with schizophrenia, according to the methods disclosed herein, can be treated as follows. The patient is administered a PANSS test and the circulating level of TNF-alpha is determined in order to determine a baseline level of cognitive and behavioral abilities, as well as detectable cytokine levels. The administration of PANSS tests and measuring blood TNF-alpha levels are well within the skilled artisans abilities when equipeed with the present disclosure and the methods herein.

The patient is then administered anti-TNF-alpha antibodies. Preferably, the activity of the antibodies is measured prior to administration to the patient, and the levels are within limits well known in the art and described herein. The anti-TNF-alpha antibodies are administered parenterally, preferably intramuscularly or intravenously to a patient. Administration takes place over a series of days, preferably two injections of antibody per day for five consecutive days. This process may be repeated based on clinical results and the patient's ability to tolerate the treatment. PANSS tests are administered at intervals following antibody administration. These results of the PANSS tests are compared to the baseline readings to evaluate progress. Further, the patient's overall well-being is monitored through other psychosocial parameters, such as sociability with other patients and medical personnel, physical activity, mood, and the like. The patient's medical condition is monitored for the appearance of rashes or allergic reactions to anti-TNF-alpha therapy. Such reactions may indicate that the treatment should be postponed, or if mild, the treatment can be continued along with therapies to alleviate rashes and allergic reactions, such as low-dose topical steroids, antihistamines, and the like. Recognition and management of rashes and other reactions are well within the abilities of one of ordinary skill in the art. PANSS scores and determination of the circulating level of TNF-alpha are monitored throughout the patient's treatment to determine the progress of the treatment. Further, continuing monitoring allows the clinician to determine if therapy is effective and if administration should continue.

The present invention also includes methods for treating psychological diseases with a combination therapy. Such diseases include, but are not limited to, schizophrenia and the like. This is because, as demonstrated by the data disclosed elsewhere herein, administration of an effective amount of an antibody to IFN-gamma and TNF-alpha results in, among other things, an improvement in a patient's mood, energy, sociability, physical activity, a decrease in negative symptoms as measured by PANSS (Positive and Negative Syndrome Scale; the standard test for evaluating schizophrenics), and a decrease in motor retardation and depression. As further demonstrated by the data disclosed herein, the administration of antibodies to IFN-gamma and TNF-alpha results in the alleviation of other symptoms associated with or mediated by schizophrenia. Such symptoms include emotional withdrawal, passive apathetic social withdrawal, will disturbance, and active social withdrawal.

The method comprises administering antibodies to IFN-gamma and TNF-alpha to a patient with schizophrenia. The antibodies are administered in an effective amount, which will be readily apparent of one of skill in the art when equipped with the present disclosure and the teachings herein. Further, the skilled clinician will be able to recognize schizophrenia when armed with the present disclosure.

As discussed elsewhere herein, an antibody comprises a polyclonal antibody, a monoclonal antibody, a humanized antibody, a camelid antibody or heavy chain antibody, and a synthetic antibody. The present invention further encompasses a biologically active fragment of an antibody, a functional equivalent of an antibody, a derivative of an antibody, an allelic variant of an antibody, and a species variant of an antibody. The antibodies, fragments, equivalents, derivatives, and variants thereof necessary to practice the methods of the present invention will be apparent to one of skill in the art when supplied with the present disclosure. The skilled artisan will further appreciate that the present invention is not limited to the singular administration of an antibody, fragment, equivalent, derivative, or variant thereof, but rather that they may be administered in a combination, either in combination with each other or in a temporal sense.

The method of the present invention further includes routes in which to administer antibodies to IFN-gamma and TNF-alpha to a patient. The skilled clinician will recognize that routes of administration may vary, depending on the status and needs of the patient, the resources available, the severity of the disease, and the like. However, as amply disclosed by the teachings provided herein, the route of administration can include, but is not limited to intramuscular, intravenous, intradermal, cutaneous, ionophoretical, topical, local, and inhalation administration. Thereby, the skilled artisan will be able to easily determine the best route of administration with little or no undue experimentation.

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### EXAMPLES

In the following examples and protocols, all commercially available reagents were utilized in accordance with the manufacturer's recommendations. The cell and protein purification methods utilized in this application are established in the art and will not be described in detail. Methodologic details may be readily derived from the cited publications.

### Example 1

### Preparation of the Immunosorbent Column

Using a column and tubing made of plastic approved for the use of blood, a column is prepared of small total volume, approximately 30-35 ml. The column is filled with immunosorbent, consisting essentially of one or more antigens or antibodies bound to Sepharose 4B or another suitable matrix, through a short filling tube placed at one end of the column. After the column has been filled, an input tube to introduce the fluid sample, and a return tube to return the treated sample to its source, are connected to either end of the column. A filter is interposed between the input tube and the column, and a second filter is interposed between the column and the return tube. The two filters prevent the flow of immunosorbent from the column. Two way stopcocks on the tubes regulate flow throughout the system.

Sepharose CL-4B (100 ml; Pharmacia, Piscataway, N.J.) is washed thoroughly with pyrogen free water, then suspended in 300 ml ice cold 1 M NaCO₃ pH 11.0. Twenty grams CNBr in 10 ml acetonitrile is added to the Sepharose. After 2 minutes this is collected on a fretted glass funnel. The Sepharose cake is washed with volumes of ice cold 0.2 M Na Bicarbonate buffer, pH 9.5, and 5 volumes of ice cold 0.5 M Na Bicarbonate buffer, pH 8.5.

The prepared Sepharose is immediately resuspended in a solution of the selected antigen or antibody or combination of one or more antigens and/or antibodies. In this case, the immunosorbent column is specifically prepared to bind to alpha IFN, so the prepared Sepharose is resuspended in a solution of 780 mg anti-alpha IFN antibody in 200 ml of 0.2 M Bicarbonate buffer, pH 9.3. This is incubated for 20 hours at 4° C. This is then centrifuged, the supernatant is decanted, and sediment is resuspended in 100 ml of 0.05 PBS (phosphate buffered saline) and 2 M glycine, pH 8.0, for 12 hours at room temperature. This is then washed thoroughly with 20 volumes of PBS.

The column is positioned lower than the source of the fluid sample, whereupon the fluid drawn from the patient flows into the column under the influence of gravity. After the fluid perfuses through the immunosorbent, it is collected in a holding tube from which it is returned to the source of the fluid.*

### Example 2

### Production of Antibody to Human Gamma IFN

Adult rabbits are immunized with purified human gamma IFN (100-106 unit/mg protein). The interferon is first mixed with equal volumes of Freund's Complete Adjuvant and 30% Arlacel A and injected IM or subcutaneously on day 1, 4, 14 and 43 (100 units, 200 units, 200, 200 respectively). Next, 200,000 units of the interferon is injected per month, for an additional 6 months. The serum is drawn from the rabbit when the titer has reached 100 units (1 unit of antibody neutralizes 10 units of gamma IFN), after which IgG is isolated and substantially purified in accordance with recognized methods.

### Example 3

### Responses to Alpha TNF, Alpha IFN, and Gamma IFN Antibodies, Separately and Together, in Patients with Active Rheumatoid Arthritis and Ankylosing Spondylitis

Polyclonal antibodies were obtained by immunizing sheep with natural human alpha IFN, and goats with recombinant human gamma IFN (“r-Hu-gamma IFN”) or recombinant human TNF-alpha (“r-Hu-TNF-alpha”), and isolating the IgG from the animals. Each milliliter of IgG contained approximately 50 mg of protein, and the antibodies showed a 1:5 signal to noise ratio at 1:1250 (anti-alpha IFN antibodies) and 1:12,500 (anti-gamma IFN antibodies and anti-alpha TNF antibodies) dilutions by ELISA (CytoImmune Sciences, Inc.). After obtaining approval and informed consent, 20 human patients with very severe rheumatoid arthritis, aged 27-64, average disease duration 9 years, were equally randomized to one of four (4) treatment groups. The patients in Group A, B and C were given one intramuscular administration of 2-3 ml/day for 5 consecutive days of (Group A) anti-alpha TNF antibodies; (Group B) anti-IFNα antibodies; or (Group C) anti-gamma IFN antibodies. The patients in Group D were given a combination of anti-TNF-alpha antibodies+anti-alpha IFN antibodies+anti-gamma IFN antibodies (6 ml/day—2 ml of each antibody). All patients met the criteria of the American College of Rheumatology for the diagnosis of RA and had not responded to any of the standard disease-modifying rheumatoid drugs. Other criteria for entry into the study included radiographic evidence of bone erosion, the presence of severe illness as indicated by the presence of 6 or more swollen joints and 3 of 4 secondary indications including 45 minutes or more of continuous morning stiffness, 6 or more painful joints, erythrocyte sedimentation rate (ESR) of 25 mm/hr or higher, and C-reactive protein of 20 mg/l or higher. Patients who were pregnant or who had serious illnesses or conditions such as anemia, leukopenia, marked ankylosis of the joints were excluded.

The primary response was determined by the Paulus index (Paulus et al., Arthritis Rheum. 33:477-484 (1990)), i.e., ≧20% or ≧50% improvement in ≧4 of 6 measures of laboratory and clinical effects (Table 2), which were obtained through day 28. These include morning stiffness, number of painful and inflamed joints, ESR, and at least a 2-point improvement on a 5-point scale of disease severity assessed by patient and by physician. To maintain consistency, the same physician was used to make all assessments.

Results

Signs of inflammation dropped in some patients within each group on day one. All groups demonstrated marked improvement by day 7, though individual variation appeared in each treatment group. Table 2 shows the proportion of patients achieving ≧20% improvement in the Paulus measures. Based on these 6 measures, the most positive response for all treatment groups was in the number of swollen and painful joints. At day 7, the positive responses using anti-TNF-alpha antibodies (Group A), and the combined antibody treatment (antibodies to all three cytokines; Group D), were the strongest. Three (3) of the five (5) patients receiving anti-TNF-alpha antibodies, and two (2) of the five (5) receiving the combined antibody treatment achieved ≧20% improvement in 4 or more Paulus measures, and at least one patient in each group achieved at least 50% improvement.

In both Group A and D, all patients had at least 20% improvement in morning stiffness and reduction in the number of painful and swollen joints. Three (3) of the five (5) patients in both groups reported at least a 2-point reduction (on a 5-point scale) in overall disease severity. At day 28, the response to anti-gamma IFN antibodies (Group C) was the strongest, including one (1) patient reporting at least 50% improvement, and two (2) others reporting at least 20% improvement in at least 4 of the 6 measures. In Group D (the combined antibody therapy), two (2) patients reported at least 20% improvement in 4 or more measures. By comparison, at day 28 only 1 of 4 patients in Group A (the anti-TNF-alpha antibody treatment group) reported having at least 20% improvement in 4 of the 6 measures. Comparable results are achieved by extracorporeal immunosorption as defined above, or by extracorporeal immunosorption in conjunction with administration of an autoimmune inhibitor.

Four (4) of the 20 patients were taken off therapy or follow-up after a temporary redness appeared at the point of injection. Two (2) patients receiving anti-1alpha IFN antibodies (Group B) and one patient each receiving anti-TNF-alpha antibodies (Group A), and the combination therapy (Group D) exhibited such reactions.

TABLE 2
Proportion of Patients Achieving ≧ 20% Improvement in Six
Measures at Day 7 and Day 28, and Paulus Index by Treatment Group

| | Anti- | | Anti- | | | | | |
|---|---|---|---|---|---|---|---|---|
| | gamma- | | alpha- | | Anti-TNF- | | | |
| Paulus | IFN | | IFN | | alpha Ab | | Combined | |
| Measures | d. 7 | d. 28 | d. 7 | d. 28 | d. 7 | d. 28 | d. 7 | d. 28 |
| Morning | 2/5 | 4/5 | 3/4 | 3/3 | 5/5 | 3/4 | 5/5 | 3/4 |
| stiffness (min.) | | | | | | | | |
| No Swollen | 4/5 | 4/5 | 2/4 | 2/3 | 5/5 | 3/4 | 5/5 | 3/4 |
| Joints | | | | | | | | |
| No Painful | 4/5 | 4/5 | 2/4 | 3/3 | 5/5 | 4/4 | 5/5 | 3/4 |
| Joints | | | | | | | | |
| Disease | 1/5 | 1/5 | 0/4 | 0/3 | 3/5 | 1/4 | 2/5 | 2/4 |
| Severity | | | | | | | | |
| (by Physician*) | | | | | | | | |
| Disease | 1/5 | 2/5 | 0/4 | 0/3 | 3/5 | 2/4 | 3/5 | 1/4 |
| Severity | | | | | | | | |
| (by Patient*) | | | | | | | | |
| ESR | 2/5 | 3/5 | 1/4 | 2/3 | 1/5 | 1/4 | 1/5 | 1/4 |
| Paulus Index | | | | | | | | |
| ≧20%** | 1/5 | 2/5 | 0/4 | 2/3 | 3/5 | 1/4 | 2/5 | 2/4 |
| ≧50%** | 0/5 | 1/5 | 0/4 | 0/3 | 1/5 | 0/4 | 1/5 | 0/4 |

*2-point improvement on 5-point scale as assessed by physician or patient.
**Proportion of patients achieving 20% (or 50%) improvement in 4 of the 6 measures at day 7 and day 28. 20% includes any patient achieving 50% improvement.

One ankylosing spondylitis (“AS”) patient, age 22, disease duration one year, was treated with the combined antibody regimen (antibodies to alpha IFN, gamma IFN, and TNF-alpha). Improvement in painful sacroiliac joint disease, diminution of radiating pain, and normalization of the erythrocyte sedimentation rate was seen on days 7-8.

For repeated treatment of human patients with autoimimune disease, or for treatment of a 30 human patient with a secondary autoimmune condition, fully humanized monoclonal antibodies must be used or, as a temporary alternative, chimeric monoclonal or multi-specied IgG polyclonal antibodies or active antibody fragment preparations.

The results indicate that a common mechanism appears to underlie all autoimmune disease, with disturbed cytokine production in different target cells producing the various clinical manifestations. Moreover, the results establish that each cytokine (e.g., alpha IFN, gamma IFN, TNF-alpha) plays its own pathological role in the mutual induction and activation of other cytokines, suggesting a single target in treatment.

Although other autoimmune diseases may require treatment with different anti-cytokines, antibodies or combination of autoimmune inhibitors, neutralization of such agents, e.g., the exemplified cytokines, appears to break the chain of pathological reactions typifying autoimmune disease and normalize the synthesis of other induced cytokines in autoimmune disease patients, including AIDS patients.

### Example 4

### Long-Term Improvement in Child with Juvenile Rheumatoid Arthritis in Response to Treatment with Gamma-IFN and TNF-Alpha Antibodies

The patient was a seven-year old girl who had been diagnosed three years earlier (January 1993) as having juvenile rheumatoid arthritis (“JRA”), polyarticular form, sero-negative, after presenting with fever, arthralgias, extreme limitation of motion in the right hip joint, neutrophilia, high ESR, and anemia. The patient improved slightly on an initial regimen of non-steroidal anti-inflammatory drugs (NSAID). Within six (6) months (Fall, 1993) exacerbation of her disease necessitated enhancing the treatment with azathioprine, NSAIDs, and with pulse therapy using Solumedrol. The patient was maintained on weekly methotrexate from February 1994 until July 1995, when her disease relapsed. However, despite increased NSAID therapy, her condition continued to deteriorate. In light of the ineffectiveness of conventional therapy, and because the disease had progressed to include hip joint involvement, which invariably leads to crippling of a child, this child became a candidate for the combined antibody treatment of the present invention.

As described above, and using immunological techniques, antibodies to gamma IFN (“anti-gamma IFN antibodies”) and antibodies to TNF-alpha (“anti-TNF-alpha antibodies”) were obtained by immunizing goats with r-gamma IFN and r-alpha INF, respectively, and isolating IgG from the immunized animals. Each milliliter of IgG contained approximately 50 mg of protein, and the antibodies showed a 1:5 signal to noise ratio at 1:12,500 dilutions by ELISA (assays performed by CytoImmune Sciences, Inc., College Park, Md.).

Two (2) ml/day each of anti-gamma IFN antibodies (3 days) and anti-TNF-alpha antibodies (5 days) were administered parenterally to the child. By the second week of observation, absence of morning stiffness, elimination of hip joint pain, and ‘considerable increases in the level of physical activity, range of motion in the affected joints, and grip strength were noted (See, Table 3). X-rays of the child showed improvement in the appearance of the femurs and hip joints, and greater delineation of articular spaces. Repeated testing of the child indicated a significant drop in disease activity, as shown by clinical and laboratory parameters, including pain, stiffness, grip strength, C-reative protein, and others (See, Table 3). The improvement in clinical status and the nearly normal range of motion in the child's hip joints persisted into the fourth month, as shown by x-rays at regular check-ups. After six months (the most recent data available), damage to the child's femurs and acetabulae were less marked as shown on x-rays, and she continued to improve in other parameters, to the point that on the advice of an orthopedist, her joints were allowed to bear greater weight.

TABLE 3
Dynamics of clinical and laboratory parameters in patient with JRA, After
treatment with Anti-gamma IFN antibodies and anti-TNF-alpha antibodies

| | Before | | | | |
|---|---|---|---|---|---|
| Parameter | Treatment | Week 1 | Week 2 | Week 3 | Week 4 |
| Arthralgia score* | 4 | 2 | 2 | 0 | 0 |
| Joint Stiffness (min.) | 30 | 10 | 0 | 0 | 0 |
| Grip Strength | 20 | 44 | 72 | 68 | 70 |
| (mm/Hg) | | | | | |
| Angle of abduction- | 15 | 15 | 20 | n/a | 30 |
| hip (degrees) | | | | | |
| Circumference of right | 12.9 | 12.7 | 12.2 | 11.9 | 12.0 |
| wrist (cm) | | | | | |
| ESC | 6 | 3 | 8 | 6 | 6 |
| Creative protein (g/l) | 0.6 | neg | neg | neg | neg |

*Scale of 0-5 where 5 is most intense pain
n/a = Not available.

These data point to a role of cytokines in autoimmune disease, and again reinforce the conclusion that a common pathological mechanism underlies clinically disparate forms of autoimmune disease. It is the differences in the target cells affected that result in the varying clinical manifestations of the autoimmune response in a patient.

As demonstrated by the results produced in this child, neutralization of certain cytokines with antibodies can break the chain of pathological reactions and normalize the synthesis of other induced cytokines in the patient. Other types of autoimmune disease can be treated by the use of anti-cytokines, singly or in combinations, to counteract autoimmune aggression and inflammation. Good results have been reported from double-blind placebo controlled trials using chimeric monoclonal anti-TNF antibodies to treat RA (Elliott et al., Lancet, 344: 1105-1110 (1994)). But until the present invention, there has been no suggestion of treatment of autoimmune disease with anti-gamma IFN antibodies, nor with a combination of anti-cytokine antibodies. Nor have the effects of such treatments been evaluated in clinical trials.

Given the striking long-term results produced by the present method, the combined anti-cytokines, e.g., anti-TNF-alpha antibodies in conjunction with anti-gamma IFN antibodies, may even act synergistically.

### Example 5

### Treatment of Patients with Systemic Lupus Erythematosus

Human patients with systemic lupus erythematosus (SLE) were selected after obtaining approval and informed consent, in much the same manner as set forth in Example 3, and divided into two groups consisting of at least four (4) patients each. The basis for selection was the patient's failure to respond to conventional therapy for SLE. Using polyclonal anti-gamma IFN antibodies and anti-TNF antibodies in accordance with Example 3, one group of patients was treated with anti-gamma IFN antibodies, while the other group was treated with anti-gamma IFN antibodies and anti-TNF antibodies. The antibodies were administered in accordance with the schedule and amounts set forth in Example 3 for 5 consecutive days.

Preliminary results, based upon at least one patient in each group, indicate that pain and swelling in joints have decreased and skin lesions have disappeared, further indicating that a common mechanism underlies all autoimmune disease, with disturbed cytokine production in different target cells producing the various clinical manifestations.

Comparable results are achieved by extracorporeal immunosorption as defined above, or by extracorporeal immunosorption in conjunction with administration of an autoimmune inhibitor.

### Example 6

### Treatment of Patients with Multiple Sclerosis

Human patients with multiple sclerosis (MS) were selected after obtaining approval and informed consent, in much the same manner as set forth in Example 3, and divided into three groups consisting of at least five (5) patients each. The basis for selection was the presence of active MS and the patient's failure to respond to conventional therapy for MS. Using polyclonal anti-gamma IFN antibodies and anti-TNF antibodies in accordance with Example 3, one group of patients was treated with anti-gamma IFN antibodies, one group with anti-TNF antibodies, and one group with anti-gamma IFN antibodies and anti-TNF antibodies. The antibodies were administered in accordance with the schedule and amounts set forth in Example 3 for 5 consecutive days, and the patients were followed for at least two and one half (2½) months.

Results of the treatment were evaluated in terms of measured neurological deficiencies and general patient function at the end of the 2¼-month period, as compared with pretreatment determinations of the same criteria. Determinations were based upon the Disability Status Scale (DSS) devised by J. F. Kurztke, and the Functional System Scale (FSS), respectively. Decreasing numbers indicate improvement on the DSS scale, while increasing numbers indicate improvement on the FSS scale. Preliminary results indicate that improvement was most evident in the group treated with anti-gamma IFN antibodies and in the group treated with anti-gamma IFN antibodies and anti-TNF antibodies, as determined by the two scales.

Additional studies indicate that the treatment may be further enhanced by the administration of beta interferon (beta IFN). When eight million international units (IU) of beta IFN were given subcutaneously to patients every ‘other day for two years, there was a decrease in the rate of exacerbated symptoms in some patients. Consequently, an optimal treatment of an MS patient appears to be the use of anti-gamma IFN antibodies or a combination of anti-gamma IFN antibodies and anti-TNF antibodies (by administration or by extracorporeal immunosorption, or both, as defined above), plus the administration of an effective amount of beta IFN.

### Example 7

### Treatment of AIDS Patients

A pilot study has been conducted with AIDS patients which indicated the correlation between a reduction in serum IFN levels and improved clinical status. In one study, four (4) patients with very high serum levels of IFN and low levels of CD4 cells (25/m³), when injected with anti-alpha IFN antibodies capable of neutralizing the circulating alpha IFN, reported an increased sense of well-being, energy, and appetite, and a disappearance of skin rashes as the circulating alpha IFN was neutralized and removed. By corollary, when the symptoms returned in one patient 5 months later, it was determined that circulating alpha IFN was again present in his blood. However, following a second cycle of treatment with anti-alpha IFN antibodies, his condition improved as the levels of circulating alpha IFN diminished. See, Skurkovich et al., Med Hypoth. 42:27-35 (1994), herein incorporated by reference.

In light of the previously demonstrated effects of reducing circulating alpha IFN in AIDS patients, and the consistently positive effect that has resulted from the combined neutralization of alpha IFN, gamma IFN and/or TNF in patients with other autoimmune diseases, similar effects are seen in AIDS patients when treated with the combined antibodies of the present invention. However, greater reduction in the clinical manifestations of AIDS disease in patients results from a combined therapy, including the neutralization or removal of alpha IFN, gamma IFN and/or TNF (by administration of antibodies to alpha IFN, gamma IFN and/or TNF, and/or their receptors, and/or by the extracorporeal exposure of the patient's fluid to an immunosorbent comprising antibodies to alpha IFN, gamma IFN and/or TNF, and/or their receptors), in conjunction with inhibition, removal or neutralization of autoimmune autoantibodies in the patient. This is accomplished by extracorporeally exposing the patient's fluid to an immunosorbent comprising CD4 cells and/or target cells in an amount sufficient to remove, neutralize or inhibit autoantibodies to CD4 cells and/or to target cells in the patient's fluid, followed by returning the fluid to the patient, in accordance with the methods disclosed herein.

### Example 8

### Treatment of Alopecia Areata

Alopecia areata is a highly unpredictable autoimmune disorder resulting in the loss of hair on the scalp and body. The disease affects about 1.7% of the world's population, including over 4 million affected in the United States. The disease is autoimmune in nature wherein the patient's hair follicles are attacked by the immune system. This results in arrest of hair growth. Alopecia areata usually presents with a small, smooth bald patch on the scalp, and can progress to total baldness.

Alopecia areata is distinct from common male pattern baldness. Because alopecia areata is an autoimmune disease, it is treatable according to the present invention, using antibody to gamma IFN.

To produce anti-IFN-gamma antibodies, goats were immunized with recombinant human IFN-gamma (Peprotech, Rocky Hill, N.J.). When titer of the anti-IFN-gamma IgG reached more than 103 IU/ml, the goats were plasmaphoresed and the IgG was isolated. F(ab′)₂ fragments were prepared by pepsin digestion and purified by gel filtration. The titer of the antibody used in the experiment described below was 24×10³ IU/ml.

To test the efficacy of an anti-gamma IFN therapy, 6 patients, ages 11 to 15 years, were treated with antibody to gamma IFN over a period of seven days. Most patients presented with lesions and baldness on the scalp, with expanding areas of baldness and hair falling out in the periphery of the lesions.

Ten intradermal injections of 0.1 milliliter of F(ab′)₂ fragments of antibody to human gamma IFN suspended in phosphate buffered saline (PBS), which were generated from goat antibodies as described above, were administered around the pathological site each day for seven days. Patients were monitored over a period of at least 8 weeks after administration of the last course of treatment. On day two of the treatment, a decrease in the amount of new hair loss was observed in two patients. On day three, four patients experienced complete cessation of new hair loss. In the no-1hair-growth areas, erythema and peri-follicular infiltration was observed, indicating that new hair growth would occur.

Four weeks after the final treatment, all patients developed thin de-pigmented hair. An additional two to four weeks later, intensive growth of normal hair in the treated lesions was observed in all patients. Minor local side-effects were experienced by the patients during about the first fifteen minutes of the therapy, but subsided. These results indicate that administration of antibody to gamma IFN to a patient with alopecia areata significantly reduces, and in most cases reverses the effects of the disease.

### Example 9

### Treatment of Vitiligo

Vitiligo is a condition that affects skin pigmentation. The cells that produce pigmentation of the skin (melanocytes) are destroyed by the person's immune system, resulting in patches of discolored, or hypopigmented skin. Vitiligo often affects the chest and abdomen, but may also affect the face around the mouth, nostrils and eyes. This condition usually occurs in people with insulin-dependent diabetes mellitus (type 1 diabetes), another autoimmune disease. To date, there is no specific treatment for vitiligo.

Anti-gamma IFN therapy was tested in vitiligo patients in the same manner as alopecia patients, but for three additional days. Four patients, ages 12-14 years old, were treated with antibody to gamma-IFN over a period of 10 days.

Ten intradermal injections of 0.1 milliliter of F(ab)₂ fragments of antibody to human gamma-IFN, which were generated from goat antibodies as described above, were administered around the pathological site each day for ten days. All four patients developed sustained erythema in the treated lesions after three days of therapy. On day five of the therapy, three patients developed small, slightly infiltrated pink papular elements in hypopigmented areas, and on day ten, all patients showed loss of well-defined borders between normal and hypopigmented skin. Minor local side-effects were experienced by the patients during about the first fifteen minutes of the therapy, but subsided. Thus, the anti-gamma IFN course of therapy resulted in production of pigmentation in the affected area.

### Example 10

### Treatment of Psoriasis

Psoriasis is a chronic skin disease characterized by periodic flare-ups of a clearly defined reddish, scaly rash that is most often located on the elbows, knees, scalp, ears, and/or lower back. Fingernails and toenails are also affected in various ways in many people with psoriasis, and approximately 10-15% of those afflicted with psoriasis will develop inflammatory arthritis. Psoriasis is characterized by an excessive proliferation of keratinocytes induced by activated CD4 Th1 lymphocytes via a complex network of cytokine interactions. However, the cause for such excessive proliferation is unclear.

Three patients, ages 9 through 13 years, were treated for seven days with antibody to gamma IFN. The protocol used here is identical to that used for alopecia therapy. Ten intradermal injections of 0.1 milliliter of F(ab)₂ fragments of antibody to human gamma-IFN, which were generated from goat antibodies as described above, were administered around the pathological site(s) each day for seven days. On day 3 of treatment, all patients experienced a marked decrease in papular infiltration and the lesions, originally ranging in size from about 5×7 centimeters to 6×12 centimeters, later became pale and no scaling was visible. After a full seven-day course of therapy, papular psoriatic lesions disappeared in all patients. Minor local side-effects were experienced by the patients during about the first fifteen minutes of the therapy, but subsided. These results indicate that antibody to gamma-IFN is an effective treatment for psoriasis and further indicates that this therapy is also an effective treatment for any skin-related autoimmune disorder.

### Example 11

### Treatment of Dystrophic Epidermolysis Bullosa

Dystrophic epidermolysis bullosa is an inherited disorder. Two forms exist, one of which is a dominant autosomally inherited disorder, the other of which is a recessive autosomally recessive disorder. Dystrophic epidermolysis bullosa results from a mutation in the gene encoding collagen type VII, the major component of anchoring fibrils. Mutations in a non-collagenous domain that catalyzes the normal antiparallel dimer formation of collagen type VII prevents dimerization, consequently an aberrant protein is generated. Humoral immune responses to the aberrant protein result in the production of autoantibodies to a key molecule in the basement membrane of the skin. This autoimmune response results in severe skin blistering, often after light contact or friction. Blistering is often present at birth; in some cases blistering is present on all skin and mucous membranes from mouth to anus. Widespread scarring is typical often leading to immobility and fusion of fingers and toes. Dystrophic epidermolysis bullosa may manifest in the gastrointestinal tract and accompanying orifices resulting in poor dentition, the inability to open the mouth fully, and esophageal webbing, resulting in malnutrition, anemia, growth retardation, and the like. Eye involvement may ensue, resulting in conjunctivitis and eyelid inflammation with adhesion to the eyeball. Genitourinary tract and respiratory tract involvement has also been noted. The prognosis of dystrophic epidermolysis bullosa is rarely positive, as malnutrition, anemia, and sepsis due to the lack of the skin barrier often claim many patients at an early age.

Dystrophic epidermolysis bullosa is distinct from many autosomal disorders in that the mutation results in an autoimmune reaction. Because dystrophic epidermolysis bullosa is an autoimmune disease, it is treatable according to the present invention, using an antibody to gamma IFN.

The following experiment was conducted which establishes that treatment of a patient having dystrophic epidermolysis bullosa with antibody to gamma interferon serves to alleviate symptoms of the disease.

A 14.5 year old male presented with dystrophic epidermolysis bullosa. The patient had visited the hospital on multiple occasions. The main symptoms were an elevated temperature (37.8° C.), bloody urine, and multiple skin blisters.

Anti-IFN-gamma antibodies were administered parenterally as described previously herein, with the exception that therapy was given twice a day for only five days. The following day, after the first administration of anti-IFN-gamma antibodies, the patient's temperature dropped to 37.1° C. without the administration of any antibiotics. Closely following treatment, the erosions and blisters on the patient's skin disappeared, and the skin epithelialized. Additionally, blood in the urine was no longer observed. Thus, a course of therapy with an antibody to gamma interferon resulted in a treatment for dystrophic epidermolysis bullosa.

### Example 12

### Treatment of Pemphigus Vulgaris

Pemphigus vulgaris is a skin disorder clinically defined as an autoimmune disease. The presence of pathogenic auto antibodies specific for desmosome proteins has been confirmed. The destruction of the desmosomes by these autoantibodies, by a complement reaction, or by other immune mediated cytological pathways results in a generalized loss of adhesion between skin cells and a loss of integrity of the skin as a whole. Certain MHC class II alleles have been linked to pemphigus vulgaris, and previous administration of thiol-containing compounds has also been linked to the disease. Other autoimmune diseases, especially myasthemia gravis and thyoma, often manifest concurrently with pemphigus vulgaris.

The incidence of pemphigus vulgaris is from about 0.5 to 3.2 patients per 100,000 people, and is most common in persons of Ashkenazi Jewish descent. Symptoms appear most often between the ages of 50 and 60, but disease onset has been described in children as well. There does not appear to be any statistically different rate of disease onset in men or women.

Pemphigus vulgaris often presents as blistering and lesion formation in the mucous membranes, especially the mouth, which may be the sole manifestation of the disease. Cutaneous lesions often follow lesions of the mucous membranes, and may appear anywhere on the body. Positive diagnosis involves immonohistology of blisters, which will demonstrate the presence of IgG₁, IgG₄, IgM or C3 on the surface of keratinocytes. Further tests for the disease include the Nikolsky sign, where firm finger-sliding pressure will cause the separation of normal appearing epidermis from the underlying tissue, and the Asboe-Hansen sign, which demonstratess that lateral pressure on a blister will casue the blister to spread to unaffected skin. Before the era of corticosteroids, the mortality rate of pemphigus vulgaris was 100%, usually due to secondary infections resulting from the lack of the defenses properties of unbroken skin, anemia, or malnutrition. Since the advent of steroid therapy, the mortality rate has dropped to 5-15%. Unfortunately, management of the disease often requires massive and constant doses of steroids, leading to osteoperosis, ocular complications, immunosuppression, malignancies, bone marrow suppression, and adrenal insufficiency. Further, present treatment regimens also include other immunosuppressive agents such as azathioprine, mycophenolate mofetil, or cyclophosphamide. In severe cases, a patient may be admitted and undergo plasmapheresis, in which the patient's serum is removed and replaced with serum that does not contain the causative antibodies.

The following experiment was conducted which establishes that treatment of a patient having pemphigus vulgaris with an antibody to interferon gamma serves to alleviate the symptoms of the disease.

A 65 year old patient presented about three years prior to the present study with a rash in the mucous membrane of the patient's mouth. The erosion was very painful and would not heal. Approximately three months after the rash appeared, the patient was hospitalized and was diagnosed with pemphigus vulgaris. Confirmation of this diagnosis was performed by the clinical laboratory, i.e., the finding of acantholytic cells on histological examination. The patient was administered 25 mg of prednisolone per day. Approximately three weeks later, the patient's health improved, the erosions epithelialized and the patient was discharged. Approximately 18 months later, erosions of approximately 3-5 cm in diameter reappeared in the mucous membrane of the mouth and later on the skin of the stomach and back. The patient returned to the clinic and a course of prednislone (30 mg per day) commenced. After stabilization, the patient was prescribed a supporting dose of prednisolone at 5 mg per day. The patient followed this prescription for approximately 18 months. In the last six weeks of the supportive prednisolone treatment, the patient's condition rapidly deteriorated with the appearance of extensive and painful erosions on the skin of the trunk and the extremities and the mucous membrane of the mouth. Prednisolone was increased to 30 mg per day to no effect. A subsequent increase to 60 mg of prednisolone per day for five days did not prevent the appearance of fresh blisters and non-healing erosions. The patient was hospitalized with a temperature of 37.2-37.8° C.

Following the failure of conventional therapy, treatment with antibodies to IFN-gamma commenced with adminsistration of 1 ml of anti-IFN-gamma antibodies intramuscularly (IM) twice a day as described elsewhere herein (IFN-gamma neutralizing activity less than or equal to about 66 mg per ml). The following day the patient's temperature normalized and the patient's general condition improved. No fresh blisters appeared. After three days of anti-IFN-gamma antibody treatment, the erosions on the mucous membrane of the mouth began to epithelialize and by the fifth day following treatment with antibodies to IFN-gamma, the erosions on the trunk area also epithelialized. In view of the stabilization of blister formation after five days of treatment with anti-IFN-gamma antibodies, the dose of prednisolone dose was gradually reduced from 25 mg per day to 5 mg per day. The patient was discharged from the hospital in satisfactory condition about three weeks days after the end of the anti-IFN-gamma antibody therapy on a supportive 5 mg per day dose of prednisolone. Thus, a course of therapy with an antibody to IFN-gamma resulted in a treatment for pemphigus vulgaris.

### Example 13

### Treatment of Schiozphrenia

A 56-year-old male patient presented with residual schizophrenia as determined by the criteria set forth in the Diagnostic and Statistical Manual of Psychiatric Disorders (4th edition, 1994, American Psychiatric Association, Washington D.C.). The patient's attire was not consistent with his age and social status and he was unkempt. The patient appeared withdrawn and bored and was spending long periods of time in bed without expressing any interest in his surroundings. His status was characterized mainly by negative symptoms of schizophrenia, such as flat effect, a decreased level of expressivity and gesticulation, emotional flatness, and no desire to be involved in social events. Cognitive disturbances were characterized by amorphous and inconcrete thought processes. The patient further demonstrated cyclothymic changes characterized by periods of hypomania and depressive symptoms. No somatic symptoms were observed upon admission.

Prior to anticytokine therapy, the patient was maintained on Haloperidol (10 mg daily) and piperidine (4 mg daily). Anticytokine therapy was started after a 7-day washout period. No psychotropic or other drugs were administered during the anticytokine therapy.

The patients blood levels of TNF-alpha was 26 pg/ml prior to therapy as determined by ELISA (R&D Systems, Minneapolis, Minn.). IFN-gamma was not detected in the patient's blood. Upon obtaining written consent from the patient, 2 mL of polyclonal anti-TNF-alpha and anti-IFN-gamma antibodies (IgG) (neutralizing activity of >66 μg/ml as determined by cell growth inhibition assays well known in the art) was administered by intramuscular injection twice daily for five successive days. The patient's clinical condition was evaluated using the PANSS test on days 0, 5, 12, 19, 26, and 34.

TABLE 4

| | DAY 0 | DAY 5 | DAY 12 | DAY 34 |
|---|---|---|---|---|
| SYMPTOM | PANSS | PANSS | PANSS | PANSS |
| Blunted Affect | 5 | 3 | 3 | 4 |
| Emotional | 5 | 2 | 2 | 3 |
| Withdrawal | | | | |
| Passive | 5 | 2 | 2 | 2 |
| Apathetic | | | | |
| Social | | | | |
| Withdrawal | | | | |
| Depression | 5 | 3 | 2 | 4 |
| Motor | 3 | 2 | 2 | 2 |
| Retardation | | | | |
| Will | 5 | 4 | 4 | 4 |
| Disturbance | | | | |
| Active Social | 3 | 2 | 2 | 2 |
| Withdrawal | | | | |

On the first evening after commencing treatment, the patient demonstrated a significant increase in energy and an improvement in mood, indicated by the patient becoming friendly, sociable and willingly entering into conversations with other patients and personnel. Subsequently his mood continued to improve, his level of physical activity, initiative and expressive abilities increased, and he demonstrated a need for activity. Significant changes were observed by the end of the first week with behavioral disturbances improving faster than cognitive abilities. The patient's clinical status was characterized by a significant decrease in negative symptoms as determined by his PANSS tests (Table 4). Subjectively, the patient became more active, lively, interested in activities on his ward and surroundings, and started to socialize with other patients. Further, his behavior became more orderly.

Twelve days after beginning anticytokine therapy the patient developed hives, but the patient's level of circulating TNF-alpha had dropped to about 1-2 pg/ml and remained low until it began to climb on day 31.

The data presented herein demonstrate, for the first time, that administration of anticytokine therapy, particularly anti-TNF-alpha antibodies, results in a clinical improvement in the symptoms characteristic of schizophrenia as measured by the PANSS test. Motor retardation, which improved from the beginning of therapy, remained unchanged throughout the entire period of observation. Will disturbance and active social withdrawal parameters decreased after the first week of therapy, and remained stable throughout the observation period. Moreover, subjective factors, such as social interaction and energy increased following administration of anti-TNF-alpha antibodies.

While not wishing to be bound by any particular theory, the data described herein demonstrate that a common mechanism underlies all autoimmune disease. Therefore, the teachings of the present invention provide methods in which the quality of life can be improved, or even extended, in patients with an autoimmune disease or condition.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1-5. (canceled)

6. A method of treating schizophrenia in a patient, the method comprising administering to the patient an effective amount of an antibody to tumor necrosis factor-alpha and an effective amount of an antibody to interferon gamma.

7. The method of claim 6, wherein the antibody is selected from the group consisting of a polyclonal antibody, a biologically active fragment thereof, an allelic variant thereof, a species variant thereof, a monoclonal antibody, a biologically active fragment thereof, an allelic variant thereof, a species variant thereof, a humanized antibody, a biologically active fragment thereof, an allelic variant thereof, a species variant thereof, a synthetic antibody, a biologically active fragment thereof, an allelic variant thereof, a species variant thereof, a heavy chain antibody, and combinations thereof.

8. The method of claim 6, wherein the antibody is administered by the route selected from the group consisting of intramuscularly, intravenously, intradermally, cutaneously, ionophoretically, topically, locally, and inhalation.

9. The method of claim 6, wherein the antibody is selected from the group consisting of a polyclonal antibody, a monoclonal antibody, a synthetic antibody, a heavy chain antibody and a humanized antibody.

10. The method of claim 9, wherein the heavy chain antibody is selected from the group consisting of a camelid antibody, a heavy chain disease antibody, and a variable heavy chain immunoglobulin.
